SEQUENCE LISTING (90 sequences)

SEQ ID NO: 1 - DNA, 22 bp, Homo sapiens
aggctgtacagggcctgctagt

SEQ ID NO: 2 - DNA, 23 bp, Homo sapiens
gccttaccttggaagtggacgta

SEQ ID NO: 3 - DNA, 27 bp, Homo sapiens
acaagtgatttttgaggagtccctatc

SEQ ID NO: 4 - DNA, 21 bp, Homo sapiens
gttcaaggagccacggcatat

SEQ ID NO: 5 - DNA, 27 bp, Homo sapiens
acaagtgatttttgaggagtccctatc

SEQ ID NO: 6 - DNA, 21 bp, Homo sapiens
gttcaaggagccacggcatat

SEQ ID NO: 7 - DNA, 23 bp, Homo sapiens
tgtcccttcagtgccctaatacc

SEQ ID NO: 8 - DNA, 22 bp, Homo sapiens
caggggagagtcttgcttaggc

SEQ ID NO: 9 - DNA, 23 bp, Homo sapiens
tgtcccttcagtgccctaatacc

SEQ ID NO: 10 - DNA, 22 bp, Homo sapiens
caggggagagtcttgcttaggc

SEQ ID NO: 11 - DNA, 23 bp, Homo sapiens
tgtcccttcagtgccctaatacc

SEQ ID NO: 12 - DNA, 22 bp, Homo sapiens
caggggagagtcttgcttaggc

SEQ ID NO: 13 - DNA, 22 bp, Homo sapiens
cgactcctgcaaacttcggtaa

SEQ ID NO: 14 - DNA, 27 bp, Homo sapiens
cttctgctgtagtacccagaacaacgg

SEQ ID NO: 15 - DNA, 22 bp, Homo sapiens
cgactcctgcaaacttcggtaa

SEQ ID NO: 16 - DNA, 27 bp, Homo sapiens
cttctgctgtagtacccagaacaacgg

SEQ ID NO: 17 - DNA, 22 bp, Homo sapiens
cgactcctgcaaacttcggtaa

SEQ ID NO: 18 - DNA, 27 bp, Homo sapiens
cttctgctgtagtacccagaacaacgg

SEQ ID NO: 19 - DNA, 21 bp, Homo sapiens
aagaagctgccgttgttctgg

SEQ ID NO: 20 - DNA, 22 bp, Homo sapiens
tcctgtaccagtctgctccgtt

SEQ ID NO: 21 - DNA, 21 bp, Homo sapiens
aagaagctgccgttgttctgg

SEQ ID NO: 22 - DNA, 22 bp, Homo sapiens
tcctgtaccagtctgctccgtt

SEQ ID NO: 23 - DNA, 22 bp, Homo sapiens
aacggagcagactggtacagga

SEQ ID NO: 24 - DNA, 23 bp, Homo sapiens
gaggtccagtgacttgttcaacg

SEQ ID NO: 25 - DNA, 22 bp, Homo sapiens
aacggagcagactggtacagga

SEQ ID NO: 26 - DNA, 23 bp, Homo sapiens
gaggtccagtgacttgttcaacg

SEQ ID NO: 27 - DNA, 22 bp, Homo sapiens
aacggagcagactggtacagga

SEQ ID NO: 28 - DNA, 23 bp, Homo sapiens
gaggtccagtgacttgttcaacg

SEQ ID NO: 29 - DNA, 22 bp, Homo sapiens
aacggagcagactggtacagga

SEQ ID NO: 30 - DNA, 23 bp, Homo sapiens
gaggtccagtgacttgttcaacg

SEQ ID NO: 31 - DNA, 22 bp, Homo sapiens
aacggagcagactggtacagga

SEQ ID NO: 32 - DNA, 23 bp, Homo sapiens
gaggtccagtgacttgttcaacg

SEQ ID NO: 33 - DNA, 21 bp, Homo sapiens
cccagctgtgtgacgttgaac

SEQ ID NO: 34 - DNA, 24 bp, Homo sapiens
gccagcacctgccccttctatgtc

SEQ ID NO: 35 - DNA, 21 bp, Homo sapiens
cccagctgtgtgacgttgaac

SEQ ID NO: 36 - DNA, 24 bp, Homo sapiens
gccagcacctgccccttctatgtc

SEQ ID NO: 37 - DNA, 21 bp, Homo sapiens
ctggttacgggtctgggtgag

SEQ ID NO: 38 - DNA, 21 bp, Homo sapiens
ggcttcagcctcagctgctac

SEQ ID NO: 39 - DNA, 22 bp, Homo sapiens
ggaggcctccacaaagacctac

SEQ ID NO: 40 - DNA, 21 bp, Homo sapiens
tatgtcctacctcccccaacg

SEQ ID NO: 41 - DNA, 22 bp, Homo sapiens
ggaggcctccacaaagacctac

SEQ ID NO: 42 - DNA, 22 bp, Homo sapiens
aggtggaaggggtgtatgtaca

SEQ ID NO: 43 - DNA, 22 bp, Homo sapiens
aggctgtacagggcctgctagt

SEQ ID NO: 44 - DNA, 23 bp, Homo sapiens
gccttaccttggaagtggacgta

SEQ ID NO: 45 - DNA, 22 bp, Homo sapiens
aggctgtacagggcctgctagt

SEQ ID NO: 46 - DNA, 23 bp, Homo sapiens
gccttaccttggaagtggacgta

SEQ ID NO: 47 - DNA, 24 bp, Homo sapiens
gaaacgtgctccacaaggtaactc

SEQ ID NO: 48 - DNA, 26 bp, Homo sapiens
cctcctcagtgtctcttagacacacc

SEQ ID NO: 49 - DNA, 24 bp, Homo sapiens
gaaacgtgctccacaaggtaactc

SEQ ID NO: 50 - DNA, 26 bp, Homo sapiens
cctcctcagtgtctcttagacacacc

SEQ ID NO: 51 - DNA, 25 bp, Homo sapiens
ggaggctctgtcaagatgttaacct

SEQ ID NO: 52 - DNA, 23 bp, Homo sapiens
tcctagggacagcaggctaagtc

SEQ ID NO: 53 - DNA, 25 bp, Homo sapiens
ggaggctctgtcaagatgttaacct

SEQ ID NO: 54 - DNA, 23 bp, Homo sapiens
tcctagggacagcaggctaagtc

SEQ ID NO: 55 - DNA, 22 bp, Homo sapiens
aaatgggtctcccttcgaaaga

SEQ ID NO: 56 - DNA, 21 bp, Homo sapiens
gggaaacctagaggtcccgag

SEQ ID NO: 57 - DNA, 22 bp, Homo sapiens
gtctgtccagtgaggagatcgg

SEQ ID NO: 58 - DNA, 22 bp, Homo sapiens
cattctcatccggaggctaggt

SEQ ID NO: 59 - DNA, 22 bp, Homo sapiens
gtctgtccagtgaggagatcgg

SEQ ID NO: 60 - DNA, 22 bp, Homo sapiens
cattctcatccggaggctaggt

SEQ ID NO: 61 - DNA, 22 bp, Homo sapiens
gtctgtccagtgaggagatcgg

SEQ ID NO: 62 - DNA, 22 bp, Homo sapiens
cattctcatccggaggctaggt

SEQ ID NO: 63 - DNA, 22 bp, Homo sapiens
ggtcctgacttgacctcgacag

SEQ ID NO: 64 - DNA, 21 bp, Homo sapiens
gagcactcagtctcggaaggg

SEQ ID NO: 65 - DNA, 22 bp, Homo sapiens
ggtcctgacttgacctcgacag

SEQ ID NO: 66 - DNA, 21 bp, Homo sapiens
gagcactcagtctcggaaggg

SEQ ID NO: 67 - DNA, 22 bp, Homo sapiens
ggtcctgacttgacctcgacag

SEQ ID NO: 68 - DNA, 21 bp, Homo sapiens
gagcactcagtctcggaaggg

SEQ ID NO: 69 - DNA, 22 bp, Homo sapiens
ggtcctgacttgacctcgacag

SEQ ID NO: 70 - DNA, 21 bp, Homo sapiens
gagcactcagtctcggaaggg

SEQ ID NO: 71 - DNA, 22 bp, Homo sapiens
agtatgagcaggggcctctagg

SEQ ID NO: 72 - DNA, 22 bp, Homo sapiens
ctggtacctgccaggtcaactc

SEQ ID NO: 73 - DNA, 22 bp, Homo sapiens
ggtggggagtagacacacctga

SEQ ID NO: 74 - DNA, 24 bp, Homo sapiens
tcttcctctcctcctttaccttgc

SEQ ID NO: 75 - DNA, 25 bp, Homo sapiens
catttcctaggtcctcatcggtaaa

SEQ ID NO: 76 - DNA, 22 bp, Homo sapiens
gagcaggtcctgcaggtcataa

SEQ ID NO: 77 - DNA, 25 bp, Homo sapiens
catttcctaggtcctcatcggtaaa

SEQ ID NO: 78 - DNA, 22 bp, Homo sapiens
gagcaggtcctgcaggtcataa

SEQ ID NO: 79 - DNA, 25 bp, Homo sapiens
catttcctaggtcctcatcggtaaa

SEQ ID NO: 80 - DNA, 22 bp, Homo sapiens
gagcaggtcctgcaggtcataa

SEQ ID NO: 81 - DNA, 27 bp, Homo sapiens
gaatgtaagaacatgacctccgtgtag

SEQ ID NO: 82 - DNA, 21 bp, Homo sapiens
tgtgtcaccaggacggaagaa

SEQ ID NO: 83 - DNA, 27 bp, Homo sapiens
gaatgtaagaacatgacctccgtgtag

SEQ ID NO: 84 - DNA, 21 bp, Homo sapiens
tgtgtcaccaggacggaagaa

SEQ ID NO: 85 - DNA, 22 bp, Homo sapiens
cagactgctgctggtattgtgc

SEQ ID NO: 86 - DNA, 21 bp, Homo sapiens
aagggaggaagatcgaatgcc

SEQ ID NO: 87 - DNA, 22 bp, Homo sapiens
ggtcaggatagatctctcagct

SEQ ID NO: 88 - DNA, 25 bp, Homo sapiens
actaatttcctcagaggctgttcaa

SEQ ID NO: 89 - DNA, 1496 bp, Homo sapiens
  CDS (39)..(1493)
agaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactgaagtgcccttcactgagagcgcctgcctgctgctgatccagcctcactatgcctctgacctggacaaggtggagggtctcactttccagcaaaactccctcaactggatgaagaaactgtctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatga

SEQ ID NO: 90 - protein, 485 aa, Homo sapiens [sequence omitted]

(19) United States
(12) Patent Application Publication
(10) Pub. No.: US 2003/0219776 A1
(43) Pub. Date: Nov. 27, 2003

(54) Molecular variants, haplotypes and linkage disequilibrium within the human angiotensinogen gene

(76) Inventors: Jean-Marc Lalouel, Salt Lake City, UT (US); Andreas Rohrwasser, Salt Lake City, UT (US); Tomoaki Ishigami, Yokohama (JP); Mitsuru Emi, Tokyo (JP); Toshiaki Nakajima, Kawasaki (JP); Ituro Inoue, Tokyo (JP)

Correspondence Address: ROTHWELL, FIGG, ERNST & MANBECK, P.C., 1425 K STREET, N.W., SUITE 800, WASHINGTON, DC, 20005, US

(21) Appl. No.: 10/321,844

(22) Filed: Dec. 18, 2002

Related U.S. Application Data
(60) Provisional application No. 60/340,482, filed on Dec. 18, 2001.

(51) Int. Cl. C12Q 1/68

(52) U.S. Cl. 435/6

(57) ABSTRACT

The present invention relates to methods for assessing risk of hypertension in als by identifying the molecular variants or haplotypes of the angiotensinogen (AGT) gene.

The present application is related to and claims priority under 35 U.S.C. §119(e) to U.S. provisional patent application Serial No. 60/340,482 filed Dec. 18, 2001.

This invention was made with Government support under Grant Nos. HL-45325 and HL-54471, awarded by the National Institutes of Health, Bethesda, Md. The United States Government has certain right in the invention.

### BACKGROUND OF THE INVENTION

The present invention relates to methods for assessing risk of hypertension in individuals by identifying the molecular variants of the angiotensinogen gene (AGT).

The publications and other materials used herein to illuminate the background of the invention, or provide additional details respecting the practice, are incorporated by reference herein, and for convenience are respectively grouped in the appended Bibliography.

Hypertension is a leading cause of human cardiovascular morbidity and mortality, with a prevalence rate of 25-30% of the adult Caucasian population of the United States (JNC Report, (1985). The primary determinants of essential hypertension, which represents 95% of the hypertensive population, have not been elucidated in spite of numerous investigations undertaken to clarify the various mechanisms involved in the regulation of blood pressure. Studies of large populations of both twins and adoptive siblings, in providing concordant evidence for strong genetic components in the regulation of blood pressure (Ward (1990)), have suggested that molecular determinants contribute to the pathogenesis of hypertension.

Among a number of factors for regulating blood pressure, the renin-angiotensin system plays an important role in salt-water homeostasis and the maintenance of vascular tone. Stimulation or inhibition of this system respectively raises or lowers blood pressure (Hall et al. (1990)) and may be involved in the etiology of hypertension. The renin-angiotensin system includes the enzymes renin and angiotensin-converting enzyme and the protein angiotensinogen (AGT). Angiotensinogen is the specific substrate of renin, an aspartyl protease. The structure of the AGT gene has been characterized (Gaillard et al. (1989); Fukamizu et al. (1990)).

Plasma angiotensinogen is primarily synthesized in the liver under the positive control of estrogens, glucocorticoids, thyroid hormones, and angiotensin II (Clauser et al. (1989)) and is secreted through the constitutive pathway. Cleavage of the amino-terminal segment of angiotensinogen by resin releases a decapeptide prohormone, angiotensin-I, which is further processed to the active octapeptide angiotensin II by the dipeptidyl carboxypeptidase angiotensin-converting enzyme (ACE). Cleavage of angiotensinogen by renin is the rate-limiting step in the activation of the renin angiotensin system (Sealey et al. (1990)). Several observations point to a direct relationship between plasma angiotensinogen concentration and blood pressure: (1) a direct positive correlation (Walker et al. (1979)); (2) high concentrations of plasma angiotensinogen in hypertensive subjects and in the offspring of hypertensive parents compared to normotensives (Fasola et al. (1968)); (3) association of increased plasma angiotensinogen with higher blood pressure in offspring with contrasted parental predisposition to hypertension (Watt et al. (1992)); (4) decreased or increased blood pressure following administration of angiotensinogen antibodies (Gardes et al. (1982)) or injection of angiotensinogen (Menard et al. (1991)); (5) expression of the angiotensinogen gene in tissues directly involved in blood pressure regulation (Campbell and Habener (1986)); and (6) elevation of blood pressure in transgenic animals overexpressing angiotensinogen (Ohkubo et al. (1990; Kimura et al. (1992)).

The etiological heterogeneity and multifactorial determination, which characterize diseases as common as hypertension, expose the limitations of the classical genetic arsenal. Definition of phenotype, model of inheritance, optimal familial structures, and candidate-gene vs. general-linkage approaches impose critical strategic choices (Lander et al. (1986; White et al. (1987; Lander et al. (1989; Lalouel (1990; Lathrop et al. (1991)). Analysis by classical likelihood ratio methods in pedigrees is problematic due to the likely heterogeneity and the unknown mode of inheritance of hypertension. While such approaches have some power to detect linkage, their power to exclude linkage appears limited. Alternatively, linkage analysis in affected sib pairs is a robust method which can accommodate heterogeneity and incomplete penetrance, does not require any a priori formulation of the mode of inheritance of the trait and can be used to place upper limits on the potential magnitude of effects exerted on a trait by inheritance at a single locus. (Blackwelder et al. (1985; Suarez et al. (1984)).

Recent studies have indicated that renin and ACE are excellent candidates for association with hypertension. The human renin gene is an attractive candidate in the etiology of essential hypertension: (1) renin is the limiting enzyme in the biosynthetic cascade leading to the potent vasoactive hormone, angiotensin II; (2) an increase in renin production can generate a major increase in blood pressure, as illustrated by renin-secreting tumors and renal artery stenosis; (3) blockade of the renin-angiotensin system is highly effective in the treatment of essential hypertension as illustrated by angiotensin I-converting enzyme inhibitors; (4) genetic studies have shown that renin is associated with the development of hypertension in some rat strains (Rapp et al. 1989; Kurtz et al. 1990); (5) transgenic animals bearing either a foreign renin gene alone (Mullins et al. 1990) or in combination with the angiotensinogen gene (Ohkubo et al. 1990) develop precocious and severe hypertension.

The human ACE gene is also an attractive candidate in the etiology of essential hypertension. ACE inhibitors constitute an important and effective therapeutic approach in the control of human hypertension (Sassaho et al. 1987) and can prevent the appearance of hypertension in the spontaneously hypertensive rat (SHR) (Harrop et al., 1990). Recently, interest in ACE has been heightened by the demonstration of linkage between hypertension and a chromosomal region including the ACE locus found in the stroke-prone SHR (Hilbert et al., 1991; Jacob et al., 1991).

Prior studies have demonstrated that the angiotensinogen gene is involved in the pathogenesis of essential hypertension. The following observations with respect to angiotensinogen and hypertension have been noted: (1) genetic linkage between essential hypertension and AGT in affected siblings; (2) association between hypertension and certain molecular variants of AGT as revealed by comparison between cases and controls; (3) increased concentrations of plasma angiotensinogen in hypertensive subjects who carry a common variant of AGT strongly associated with hypertension; (4) persons with the most common AGT gene variant exhibit only raised levels of plasma angiotensinogen and high blood pressure; and (5) the most common AGT gene variant has been found to be statistically increased in women presenting preeclampsia during pregnancy, a condition occurring in 5-10% of all pregnancies. The association between renin, ACE or AGT and essential hypertension was studied using the affected sib pair method (Bishop et al. (1990)) on populations from Salt Lake City, Utah and Paris, France, as described in further detail in the Examples. Only an association between the AGT gene and hypertension was found. The AGT gene was examined in persons with hypertension, and at least 15 variants have been identified. None of these variants occur in the region of the AGT protein cleaved by either renin or ACE. Identification of the AGT gene as being associated with essential hypertension was confirmed in a population study of healthy subjects and in women presenting preeclampsia during pregnancy. See, e.g., U.S. Pat. Nos. 5,374,525 and 5,763,168, each incorporated herein by reference; U.S. patent application Ser. No. 09/106,216, filed Jun. 29, 1998, incorporated herein by reference; Jeunemaitre et al. (1992); Jeunemaitre et al. (1993); and Jeunemaitre et al. (1997).

According to Gaillard et al. (1989), the human AGT gene contains five exons and four introns which span 13 Kb. The first exon (37 bp) codes for the 5′ untranslated region of the MRNA. The second exon codes for the signal peptide and the first 252 amino acids of the mature protein. Exons 3 and 4 are shorter and code for 90 and 48 amino acids, respectively. Exon 5 contains a short coding sequence (62 amino acids) and the 3′-untranslated region. Genbank accession No. AH002594 also sets forth a sequence of the AGT gene as revised on Oct. 30, 1994. The revised sequence moves the start site of transcription one nucleotide 5′ of the transcription start site identified in Gaillard et al. (1989). Since polymorphisms described herein and in the prior art have been written with respect to the Gaillard et al. (1989) transcription start site, this nomenclature will also be used herein.

Much attention is now focused on the identification of susceptibility genes underlying complex diseases through whole-genome linkage disequilibrium (LD) mapping with single nucleotide polymorphisms (SNPs). The feasibility of such studies is currently under debate and depends explicitly on the persistence of LD between SNPs and causal mutations (Collins et al. 1997; Jorde 2000; Kruglyak 1999; Pritchard and Przeworski 2001; Risch and Merikangas 1996; Risch 2000). The ability to detect LD within a given genomic region depends on several factors. Recombination rates vary by more than an order of magnitude across the genome (Yu et al. 2001), creating substantial variation in LD levels in different genomic regions (Huttley et al. 1999; Pritchard and Przeworski 2001; Reich et al. 2001; Taillon-Miller et al. 2000). Furthermore, the extent of LD varies considerably among different populations, reflecting the effects of population structure and history (Kidd et al. 2000; Kidd et al. 1998; Laan and Paabo 1997; Tishkoff et al. 1998; Tishkoff et al. 2000; Zavattari et al. 2000). Finally, the presence of several disease-predisposing alleles within a susceptibility locus, each in association with a different background haplotype, can seriously compromise the ability of LD to locate the susceptibility locus (Xiong and Guo 1998). Considering the potential effects of these and other factors, it is not surprising that simulations and empirical studies have arrived at highly disparate results regarding the expected extent of LD in the human genome and the resultant SNP density required for successful LD studies (Abecasis et al. 2001; Bonnen et al. 2000; Collins et al. 1999; Eaves et al. 2000; Jorde 1995; Kruglyak 1999; Moffatt et al. 2000; Reich et al. 2001; Stephens et al. 2001). Because of their important implications for the design of gene mapping studies, these issues need to be resolved with additional empirical data.

AGT represents one of the few genes in which genetic variation has been shown to be associated with measurable variation in an endophenotype (plasma angiotensinogen) and in a biomedically relevant phenotype, hypertension (Jeunemaitre et al. 1992). In previous studies, it has been reported that two common polymorphisms, T235M and A−6G, are significantly associated with essential hypertension (EHT) (MIM 145500) (Inoue et al. 1997; Jeunemaitre et al. 1997). The T235 allele is in nearly complete LD with A(−6) and is associated with higher plasma angiotensinogen levels. These results have been replicated in many other studies (Iso et al. 2000; Pan et al. 2000; Rankinen et al. 2000; Rice et al. 2000; Sato et al. 2000), but not all (Bengtsson et al. 1999; Brand et al. 1998; Kato et al. 2000; Larson et al. 2000; Niu et al. 1999; Province et al. 2000; Taittonen et al. 1999). This inconsistency may reflect differences in phenotype definition, lack of statistical power, population history or structure, the effects of other loci, and the varying effects of several disease-predisposing variants within A GT (Corvol et al. 1999; Lalouel 2001). Nevertheless, several major meta-analyses have confirmed a significant association between AGT variation and hypertension, with a combined relative risk of approximately 1.2 for the T235 allele (Kato et al. 1999; Kunz et al. 1997; Staessen et al. 1999). AGT thus represents an important locus whose variation is involved in the predisposition to a common disease

It is an object of the present invention to identify additional AGT polymorphisms associated with hypertension and to utilize such polymorphisms for determining predisposition to hypertension in individuals. It is a further object of the present invention to evaluate methods for assessing risk of hypertension by investigating the molecular variants of the angiotensinogen gene. Identification of individuals who may be predisposed to hypertension will lead to better management of the disease, since diagnosis of predisposition can help influence course of treatment for hypertension in affected individuals.

### SUMMARY OF THE INVENTION

The present invention relates to methods for determining the predisposition of an individual to hypertension by analyzing the DNA sequence of the angiotensinogen gene of the individual for molecular variants of the angiotensinogen gene. Such methods can be used inter alia in diagnosing a predisposition to hypertension in an individual.

More specifically, the present invention relates to identification of additional polymorphisms of the AGT gene associated with human hypertension. The analysis of the AGT gene for these polymorphisms will identify subjects with a genetic predisposition to develop essential hypertension or pregnancy-induced hypertension. The management of hypertension in these subjects could then be more specifically managed, e.g., by dietary sodium restriction, by carefully monitoring blood pressure and treating with conventional drugs, by the administration of renin inhibitors or by the administration of drugs to inhibit the synthesis of AGT. The analysis of the AGT gene is performed by comparing the DNA sequence of an individual's AGT gene with the DNA sequence of the native, non-variant A GT gene.

In one embodiment, the invention provides several new polymporphisms as described herein that can be can be used to determine the predisposition to hypertension. It has further been found that some of these polymorphisms occur in linkage disequilibrium with the variants M/T(235), G/A(−6), and other molecular variants, as described in further detail herein. Accordingly, in another embodiment the invention provides a method of that which can be used in place of, or in addition to, an analysis based upon the previously known molecular variants.

DNA sequencing of the entire angiotensinogen gene (AGT) in a series of Japanese and Caucasian study subjects has led to the identification of 44 single nucleotide polymorphisms (SNPs) in the AGT gene. Typing of 21 of these SNPs in larger series of subjects has afforded the definition of the haplotype structure of the gene, that is, the observed distribution of these genetic variants on human chromosomes. These data document that the six most common haploytpes are sufficient to describe the majority of the variation observed in the AGT gene in either population. Thus, in another embodiment the invention provides a reduced set of SNPs that can be used to characterize such haplotypes by conventional DNA typing methods. Further evaluation of this variation aids in assessing predisposition for hypertension. Significant LD is found between susceptibility alleles in the AGT region and other SNP's. The analysis of the AGT gene for molecular variants will identify subjects with a genetic predisposition to develop essential hypertension or pregnancy-induced hypertension.

The present invention also relates to the identification of haplotypes of the AGT gene which can also be used to determine predisposition to hypertension. In accordance with this aspect of the present invention, the haplotype of an individual is analyzed for the alleles described herein and the presence of a particular haplotype is then associated with a predisposition to hypertension.

### BRIEF DESCRIPTION OF THE FIGURES

FIGS. 1A-1C show a schematic diagram of AGT showing the locations of the five exons (FIG. 1A), repeat elements (FIG. 1B) and 44 SNPs identified (FIG. 1C). The complete genome sequence containing entire AGT spaced 14.4 kb (10.1% coding sequence) was determined. The exact sizes of intron 1, 2, 3, and 4 are 3233 bp, 3794 bp, 1595 bp, and 863 bp, respectively (FIG. 1A). Repetitive elements (SINE, LINE, and LTR), simple repeats elements were analyzed with RepeatMasker (http://ftp.genome.washington.edu/RM/RepeatMasker.html). The location of the dinucleotide repeat sequence is shown in FIG. 1B. Forty-four (44) SNPs were identified and the locations of SNPs are shown in FIG. 1C.

FIGS. 2A and 2B show the LD between T235M and other SNPs in AGT. Pair-wise LD between T235M and other SNPs evaluated by either D′ (FIG. 2A) or r² (FIG. 2B) in Caucasians and Japanese. D′ is expressed as an absolute value.

FIGS. 3A-3D show comparisons of LD versus physical distance between all SNPs in a pair-wise fashion. The relationships between LD and physical distance based on the 861 marker pairs in Japanese individuals are shown. Pair-wise LD, evaluated by either D′ (FIG. 3A) or r² (FIG. 3B), was plotted against physical distance between the SNPs. Average values of D′ (FIG. 3C) and r² (FIG. 3D) at every 500 bp in. Caucasians and Japanese show that LD declines with increasing physical distance between SNP pairs.

FIGS. 4A and 4B show pair-wise LD in AGT evaluated by r². LD between all pairs of SNPs (SNPᵢ and SNPⱼ, where i and j are referred to SNP number in Table 2) was evaluated by the LD measure, r². Pair-wise LD was determined among the 861 marker pairs studied in Caucasians (FIG. 4A) and Japanese (FIG. 4B) and pairs in LD (r²●0.5) are shown as black boxes (●). Several SNPs created subgroups in which SNPs were in tight LD each other. The subgroup was shown in the bottom. A dot in the center of square indicated no data, because SNP24 and SNP27 were not observed in Caucasians.

FIG. 5 shows AGT haplotypes in Caucasians and Japanese. These haplotypes were constructed and the frequencies were estimated by the EM algorithm based on twenty-one SNPs in AGT. Black box shows the minor allele in Japanese. The chimpanzee sequence is also shown.

FIG. 6 shows a plot of DSS (y axis), the difference in the sum of squares between trees generated from two halves of a 1500 bp sliding window of DNA sequence against the position of the center of each sliding window (x axis). Gaps in the sequence represent those portions of the sequence in which no polymorphic variation was present.

FIGS. 7A and 7B show haplotype trees for AGT haplotype based on twenty-one SNPs and the chimpanzee sequence. The size of each circles indicated the frequencies of haplotypes in Caucasians (FIG. 7A) and Japanese (FIG. 7B).

FIGS. 8A-8C show relationships between four major SNP haplotypes and the microsatellite marker. The distribution of the frequency of individual microsatellite alleles is shown for each of the common SNP haplotypes in AGT. Even though the distribution of CA-repeat allele (FIG. 8A) is very different between Caucasian and Japanese, each SNP haplotype was associated with a specific allele of CA-repeat in Caucasians (FIG. 8B) and Japanese (FIG. 8C).

### SUMMARY OF THE SEQUENCE LISTING

SEQ ID NOs:1 and 2 are 2 oppositely oriented oligonucleotides used to screen the PAC library. SEQ ID Nos: 3-88 are overlapping primer sets covering the genome sequence of AGT. They were designed on the basis of size and overlap of PCR amplicons. SEQ ID NO:89 sets forth a wild-type cDNA sequence of the AGT gene according to Gaillard et al. (1989). SEQ ID NO:90 sets forth the corresponding protein sequence for this cDNA sequence.

### DETAILED DESCRIPTION OF THE INVENTION

The present invention is directed to methods for assessing predisposition of hypertension by investigating the variants in the angiotensinogen gene. The present invention has found that variation in the angiotensinogen gene is caused by 6 major haplotypes. In order to understand this genetic variation, a 14.4 kb region spanning the entire AGT gene was sequenced and 44 SNPs were identified. SNP's were identified and analyzed using techniques well known in the art and also as described in Nakajima, et al., Am J Hum Genet 2002 Jan; 70(1):108-23. By analyzing the DNA sequence of the angiotensinogen gene for SNPs disclosed herein, or alternatively for the haplotypes disclosed herein, the predisposition of an individual to hypertension can be identified.

Because variation in AGT has been shown to correlate with variation in plasma angiotensinogen and risk of hypertension, AGT provides the basis for a useful study of LD patterns in a locus that helps to determine susceptibility to hypertension.

The analysis of the AGT gene for LD will identify subjects with a genetic predisposition to develop essential hypertension or pregnancy-induced hypertension. The management of hypertension in these subjects could then be more specifically managed, e.g., by dietary sodium restriction, by carefully monitoring blood pressure and treating with conventional drugs, by the administration of renin inhibitors or by the administration of drugs to inhibit the synthesis of AGT. The analysis of the AGT gene is performed by comparing the DNA sequence of an individual's AGT gene with the DNA sequence of the native, non-variant AGT gene. It has been found that an analysis of the AGT gene intron 1, specifically nucleotide position 67 relative to the transcription start site of Gaillard et al. (1989) of the AGT gene sequence described in further detail herein, can be used to determine the predisposition to hypertension. It has further been found that this polymorphism occurs in linkage disequilibrium with the M/T(235), G/A(−6), and other molecular variants, as described in further detail herein. Accordingly, analysis of this polymorphism can be used in place of an analysis of the latter molecular variants.

The identification of the association between the AGT gene and hypertension permits the screening of individuals to determine a predisposition to hypertension. Those individuals who are identified at risk for the development of the disease may benefit from dietary sodium restriction, can have their blood pressure more closely monitored and be treated at an earlier time in the course of the disease. Such blood pressure monitoring and treatment may be performed using conventional techniques well known in the art.

To identify persons having a predisposition to hypertension, the variants of the AGT gene were investigated. Genomic DNA from 77 Japanese individuals was collected. The PAC/BAC clone and genome sequence of human and chimpanzee AGT was isolated. Next, SNPs were identified by subjecting genomic DNA to PCR amplification, followed by sequencing. By comparing the sequences from 72 chromosomes, polymorphisms were identified. The data was then subjected to statistical analysis.

In order to analyze the molecular variants in AGT, first, a 14.4 kb genomic region containing the entire AGT gene was sequenced. Known repetitive elements were used for early linkage studies. Forty-four (44) SNPs were identified in the total of 72 chromosomes. The subjects were then genotyped for each of the 44 SNPs.

LD between T235M and other SNPs were studied because of the reported association between the T235 allele and EHT. The results demonstrated that significant LD is found between susceptibility alleles.

In one aspect, the invention provides probes and primers for use in a prognostic or diagnostic assay. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence of the AGT gene, including 5′ and/or 3′ untranslated regions. In preferred embodiments, the probe further comprises a label group attached thereto wherein the label can be detected as an indicator for the presence of the probe, e.g., the label group can be selected from amongst radioisotopes, fluorescent compounds, enzymes, and enzyme co-factors.

In a further aspect, the present invention features methods for determining whether a subject is at risk for developing hypertension. According to the diagnostic and prognostic methods of the present invention, alteration of the wild-type AGT locus is detected. “Alteration of a wild-type gene” encompasses all forms of mutations including deletions, insertions and point mutations in the coding and noncoding regions. Deletions may be of the entire gene or of only a portion of the gene. Point mutations may result in stop codons, frameshift mutations or amino acid substitutions. Point mutations or deletions in the promoter can change transcription and thereby alter the gene function. Somatic mutations are those which occur only in certain tissues and are not inherited in the germline. Germline mutations can be found in any of a body's tissues and are inherited. The finding of AGT germline mutations thus provides diagnostic information. An AGT allele which is not deleted (e.g., found on the sister chromosome to a chromosome carrying an AGT deletion) can be screened for other mutations, such as insertions, small deletions, and point mutations. Point mutational events may occur in regulatory regions, such as in the promoter of the gene, or in intron regions or at intron/exon junctions.

Useful diagnostic techniques include, but are not limited to fluorescent in situ hybridization (FISH), direct DNA sequencing, PFGE analysis, Southern blot analysis, single stranded conformation analysis (SSCA), RNase protection assay, allele-specific oligonucleotide (ASO), dot blot analysis and PCR-SSCP, as discussed in detail further below. Also useful is the recently developed technique of DNA microchip technology. In addition to the techniques described herein, similar and other useful techniques are also described in U.S. Pat. Nos. 5,837,492 and 5,800,998, each incorporated herein by reference.

Predisposition to disease can be ascertained by testing any tissue of a human for mutations of the AGT gene. For example, a person who has inherited a germline AGT mutation would be prone to develop hypertension. This can be determined by testing DNA from any tissue of the person's body. Most simply, blood can be drawn and DNA extracted from the cells of the blood. In addition, prenatal diagnosis can be accomplished by testing fetal cells, placental cells or amniotic cells for mutations of the AGT gene. Alteration of a wild-type AGT allele, whether, for example, by point mutation or deletion, can be detected by any of the means discussed herein.

There are several methods that can be used to detect DNA sequence variation. Direct DNA sequencing, either manual sequencing or automated fluorescent sequencing can detect sequence variation. Another approach is the single-stranded conformation polymorphism assay (SSCA) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments which have shifted mobility on SSCA gels are then sequenced to determine the exact nature of the DNA sequence variation. Other approaches based on the detection of mismatches between the two complementary DNA strands include clamped denaturing gel electrophoresis (CDGE) (Sheffield et al., 1991), heteroduplex analysis (HA) (White et al., 1992) and chemical mismatch cleavage (CMC) (Grompe et al., 1989). None of the methods described above will detect large deletions, duplications or insertions, nor will they detect a regulatory mutation which affects transcription or translation of the protein. Other methods which might detect these classes of mutations such as a protein truncation assay or the asymmetric assay, detect only specific types of mutations and would not detect missense mutations. A review of currently available methods of detecting DNA sequence variation can be found in a recent review by Grompe (1993). Once a mutation is known, an allele specific detection approach such as allele specific oligonucleotide (ASO) hybridization can be utilized to rapidly screen large numbers of other samples for that same mutation.

Detection of point mutations can be accomplished by molecular cloning of the AGT allele(s) and sequencing the allele(s) using techniques well known in the art. Alternatively, the gene sequences can be amplified directly from a genomic DNA preparation from the tissue, using known techniques. The DNA sequence of the amplified sequences can then be determined.

There are six well known methods for a more complete, yet still indirect, test for confirming the presence of a susceptibility allele: 1) single-stranded conformation analysis (SSCA) (Orita et al., 1989); 2) denaturing gradient gel electrophoresis (DGGE) (Wartell et al., 1990; Sheffield et al., 1989); 3) RNase protection assays (Finkelstein et al., 1990; Kinszler et al., 1991); 4) allele-specific oligonucleotides (ASOs) (Conner et al., 1983); 5) the use of proteins which recognize nucleotide mismatches, such as the E. coli mutS protein (Modrich, 1991); and 6) allele-specific PCR (Rano and Kidd, 1989). For allele-specific PCR, primers are used which hybridize at their 3′ ends to a particular AGT mutation. If the particular AGT mutation is not present, an amplification product is not observed. Amplification Refractory Mutation System (ARMS) can also be used, as disclosed in European Patent Application Publication No. 0332435 and in Newton et al., 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the AGT mutation found in that individual. Other techniques for detecting insertions and deletions as known in the art can be used.

In the first three methods (SSCA, DGGE and RNase protection assay), a new electrophoretic band appears. SSCA detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of tumor samples. An example of a mismatch cleavage technique is the RNase protection method. In the practice of the present invention, the method involves the use of a labeled riboprobe which is complementary to the human wild-type AGT gene coding sequence. The riboprobe and either mRNA or DNA isolated from the tumor tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the AGT mRNA or gene but can be a segment of either. If the riboprobe comprises only a segment of the AGT mRNA or gene, it will be desirable to use a number of these probes to screen the whole MRNA sequence for mismatches.

In similar fashion, DNA probes can be used to detect mismatches, through enzymatic or chemical cleavage. See, e.g., Cotton et al., 1988; Shenk et al., 1975; Novack et al., 1986. Alternatively, mismatches can be detected by shifts in the electrophoretic mobility of mismatched duplexes relative to matched duplexes. See, e.g., Cariello, 1988. With either riboprobes or DNA probes, the cellular mRNA or DNA which might contain a mutation can be amplified using PCR before hybridization. Changes in DNA of the AGT gene can also be detected using Southern hybridization, especially if the changes are gross rearrangements, such as deletions and insertions.

DNA sequences of the AGT gene which have been amplified by use of PCR may also be screened using allele-specific probes. These probes are nucleic acid oligomers, each of which contains a region of the AGT gene sequence harboring a known mutation. For example, one oligomer may be about 30 nucleotides in length (although shorter and longer oligomers are also usable as well recognized by those of skill in the art), corresponding to a portion of the AGT gene sequence. By use of a battery of such allele-specific probes, PCR amplification products can be screened to identify the presence of a previously identified mutation in the AGT gene. Hybridization of allele-specific probes with amplified AGT sequences can be performed, for example, on a nylon filter. Hybridization to a particular probe under high stringency hybridization conditions indicates the presence of the same mutation in the tumor tissue as in the allele-specific probe.

The newly developed technique of nucleic acid analysis via microchip technology is also applicable to the present invention. In this technique, thousands of distinct oligonucleotide probes are built up in an array on a silicon chip. Nucleic acid to be analyzed is fluorescently labeled and hybridized to the probes on the chip. It is also possible to study nucleic acid-protein interactions using these nucleic acid microchips. Using this technique one can determine the presence of mutations or even sequence the nucleic acid being analyzed or one can measure expression levels of a gene of interest. The method is one of parallel processing of many, even thousands, of probes at once and can tremendously increase the rate of analysis. Several papers have been published which use this technique. Some of these are Hacia et al., 1996; Shoemaker et al., 1996; Chee et al., 1996; Lockhart et al., 1996; DeRisi et al., 1996; Lipshutz et al., 1995. This method has already been used to screen people for mutations in the breast cancer gene BRCA1 (Hacia et al., 1996). This new technology has been reviewed in a news article in Chemical and Engineering News (Borman, 1996) and been the subject of an editorial (Nature Genetics, 1996). Also see Fodor (1997).

The most definitive test for mutations in a candidate locus is to directly compare genomic A GT sequences from disease patients with those from a control population. Alternatively, one could sequence messenger RNA after amplification, e.g., by PCR, thereby eliminating the necessity of determining the exon structure of the candidate gene.

Mutations from disease patients falling outside the coding region of AGT can be detected by examining the non-coding regions, such as introns and regulatory sequences near or within the AGT gene. An early indication that mutations in noncoding regions are important may come from Northern blot experiments that reveal messenger RNA molecules of abnormal size or abundance in disease patients as compared to control individuals.

Alteration of AGT mRNA expression can be detected by any techniques known in the art. These include Northern blot analysis, PCR amplification and RNase protection. Diminished or increased mRNA expression indicates an alteration of the wild-type AGT gene. Alteration of wild-type AGT genes can also be detected by screening for alteration of wild-type AGT protein. For example, monoclonal antibodies immunoreactive with AGT can be used to screen a tissue. Lack of cognate antigen would indicate an AGT mutation. Antibodies specific for products of mutant alleles could also be used to detect mutant AGT gene product. Such immunological assays can be done in any convenient formats known in the art. These include Western blots, immunohistochemical assays and ELISA assays. Any means for detecting an altered AGT protein can be used to detect alteration of wild-type AGT genes. Functional assays, such as protein binding determinations, can be used. In addition, assays can be used which detect AGT biochemical function. Finding a mutant AGT gene product indicates alteration of a wild-type AGT gene.

The primer pairs of the present invention are useful for determination of the nucleotide sequence of a particular AGT allele using PCR. The pairs of single-stranded DNA primers can be annealed to sequences within or surrounding the AGT gene on chromosome 12 in order to prime amplifying DNA synthesis of the AGT gene itself. A complete set of these primers allows synthesis of all of the nucleotides of the AGT gene coding sequences, i.e., the exons. The set of primers preferably allows synthesis of both intron and exon sequences. Allele-specific primers can also be used. Such primers anneal only to particular AGT mutant alleles, and thus will only amplify a product in the presence of the mutant allele as a template.

In order to facilitate subsequent cloning of amplified sequences, primers may have restriction enzyme site sequences appended to their 5′ ends. Thus, all nucleotides of the primers are derived from AGT sequences or sequences adjacent to AGT, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the known sequences of the A GT exons, the design of particular primers is well within the skill of the art. Suitable primers for mutation screening are also described herein.

The nucleic acid probes provided by the present invention are useful for a number of purposes. They can be used in Southern hybridization to genomic DNA and in the RNase protection method for detecting point mutations already discussed above. The probes can be used to detect PCR amplification products. They may also be used to detect mismatches with the AGT gene or mRNA using other techniques.

The alleles of the AGT gene in an individual to be tested are cloned using conventional techniques. For example, a blood sample is obtained from the individual. The genomic DNA isolated from cells in this sample is partially digested to an average fragment size of approximately 20 kb. Fragments in the range from 18-21 kb are isolated. The resulting fragments are ligated into an appropriate vector. The sequences are then analyzed as described above.

Alternatively, polymerase chain reactions (PCRs) are performed with primer pairs for the 5′ region or the exons of the AGT gene. Examples of such primer pairs are set forth in U.S. Pat. No. 5,374,525, U.S. Pat. No. 6,153,386 and herein in Table 1. PCRs can also be performed with primer pairs based on any sequence of the normal AGT gene. For example, primer pairs for the large intron can be prepared and utilized. Finally, PCR can also be performed on the mRNA. The amplified products are then analyzed as described above.

### EXAMPLES

The present invention is further detailed in the following Examples, which are offered by way of illustration and are not intended to limit the invention in any manner. Standard techniques well known in the art or the techniques specifically described below, or in U.S. Pat. No. 5,374,525 or in U.S. Pat. No. 6,153,386 are utilized.

### Example 1

### Materials and Methods for DNA Analysis

Subjects: Seventy-seven Japanese individuals unselected for disease status were recruited from out-patient clinics at Yokohama City University Hospital. Informed consent was obtained from each subject, and the study was performed with the approval of the Ethical Committee of Yokohama City University. Blood samples were collected for isolation of genomic DNA. The 88 Caucasian subjects are unrelated individuals from the Utah subset of the CEPH collection.

Isolation of PAC/BAC clone and genome sequence of human and Chimpanzee AGT: A bacteriophage P1-derived artificial chromosome (PAC) library containing human genomic DNA pooled in a three-dimensional structure (Genome Systems, Inc., St. Louis, Mo.) was screened for the AGT clone. The PAC library was screened by the method previously described using two oppositely oriented oligonucleotides

| | |
|---|---|
| 5′-AGGCTGTACAGGGCCTGCTAGT-3′ | (SEQ ID NO: 1) |
| 5′-GCCTTACCTTGGAAGTGGACGTA-3. | (SEQ ID NO:2) |

A high-density hybridization filter for chimpanzee genomic DNA is available from BAC/PAC Resources, Children's Hospital Oakland Research Institute. The filters were hybridized with digoxigenin-labeled (randomly primed, Roche) probes on exon 2 of AGT. E. coli bearing the clones was cultured and BAC/PAC DNA was isolated as described previously (Nakajima et al. 2000).

Promoter and exon sequences were obtained from GenBank (accession number NM_{—}000029 and X15323). Intron sequences were determined from a PAC genome clone containing AGT by direct primer walking across the gaps. Sequencing was performed by BigDye Terminator cycle sequencing using an ABI 377 Prism automated DNA sequencer (Applied Biosystems, Tokyo, Japan). Interspersed repeats in the gene were identified by RepeatMasker.

Identification of single nucleotide polymorphisms: Overlapping primer sets covering the genome sequence of AGT were designed on the basis of size and overlap of PCR amplicons (Table 1). Genomic DNA was subjected to PCR amplification followed by sequencing using the BigDye Terminator cycle. Polymorphisms were identified by the comparison of sequences from 72 chromosomes (36 from Japanese and 36 from Caucasians) using the Sequencher™ program (Gene Code Co., Ann Arbor, Mich., USA). Each polymorphism has been confirmed by reamplifying and resequencing from the same or the opposite strand. The remainder of the study subjects were sequenced only for the regions in which SNPs were identified in the first set of 72 chromosomes.

TABLE 1

Oligonucleotide Primers for SNP Genotyping in the Hunmn AGT

| | | |
|---|---|---|
| SNP No. | Upstream Primer (SEQ ID NO:) | Downstream Primer (SEQ ID NO:) |

| | | |
|---|---|---|
| 1 | ACAAGTGATTTTTGAGGAGTCCCTATC (3) | GTTCAAGGAGCCACGGCATAT (4) |
| 2 | ACAAGTGATTTTTGAGGAGTCCCTATC (5) | GTTCAAGGAGCCACGGCATAT (6) |
| 3 | TGTCCCTTCAGTGCCCTAATACC (7) | CAGGGGAGAGTCTTGCTTAGGC (8) |
| 4 | TGTCCCTTCAGTGCCCTAATACC (9) | CAGGGGAGAGTCTTGCTTAGGC (10) |
| 5 | TGTCCCTTCAGTGCCCTAATACC (11) | CAGGGGAGAGTCTTGCTTAGGC (12) |
| 6 | CGACTCCTGCAAACTTCGGTAA (13) | CTTCTGCTGTAGTACCCAGAACAACGG (14) |
| 7 | CGACTCCTGCAAACTTCGGTAA (15) | CTTCTGCTGTAGTACCCAGAACAACGG (16) |
| 8 | CGACTCCTGCAAACTTCGGTAA (17) | CTTCTGCTGTAGTACCCAGAACAACGG (18) |
| 9 | AAGAAGCTGCCGTTGTTCTGG (19) | TCCTGTACCAGTCTGCTCCGTT (20) |
| 10 | AAGAAGCTGCCGTTGTTCTGG (21) | TCCTGTACCAGTCTGCTCCGTT (22) |
| 11 | AACGGAGCAGACTGGTACAGGA (23) | GAGGTCCAGTGACTTGTTCAACG (24) |
| 12 | AACGGAGCAGACTGGTACAGGA (25) | GAGGTCCAGTGACTTGTTCAACG (26) |
| 13 | AACGGAGCAGACTGGTACAGGA (27) | GAGGTCCAGTGACTTGTTCAACG (28) |
| 14 | AACGGAGCAGACTGGTACAGGA (29) | GAGGTCCAGTGACTTGTTCAACG (30) |
| 15 | AACGGAGCAGACTGGTACAGGA (31) | GAGGTCCAGTGACTTGTTCAACG (32) |
| 16 | CCCAGCTGTGTGACGTTGAAC (33) | GCCAGCACCTGCCCCTTCTATGTC (34) |
| 17 | CCCAGCTGTGTGACGTTGAAC (35) | GCCAGCACCTGCCCCTTCTATGTC (36) |
| 18 | CTGGTTACGGGTCTGGGTGAG (37) | GGCTTCAGCCTCAGCTGCTAC (38) |
| 19 | GGAGGCCTCCACAAAGACCTAC (39) | TATGTCCTACCTCCCCCAACG (40) |
| 20 | GGAGGCCTCCACAAAGACCTAC (41) | AGGTGGAAGGGGTGTATGTACA (42) |
| 21 | AGGCTGTACAGGGCCTGCTAGT (43) | GCCTTACCTTGGAAGTGGACGTA (44) |
| 22 | AGGCTGTACAGGGCCTGCTAGT (45) | GCCTTACCTTGGAAGTGGACGTA (46) |
| 23 | GAAACGTGCTCCACAAGGTAACTC (47) | CCTCCTCAGTGTCTCTTAGACACACC (48) |
| 24 | GAAACGTGCTCCACAAGGTAACTC (49) | CCTCCTCAGTGTCTCTTAGACACACC (50) |
| 25 | GGAGGCTCTGTCAAGATGTTAACCT (51) | TCCTAGGGACAGCAGGCTAAGTC (52) |
| 26 | GGAGGCTCTGTCAAGATGTTAACCT (53) | TCCTAGGGACAGCAGGCTAAGTC (54) |
| 27 | AAATGGGTCTCCCTTCGAAAGA (55) | GGGAAACCTAGAGGTCCCGAG (56) |
| 28 | GTCTGTCCAGTGAGGAGATCGG (57) | CATTCTCATCCGGAGGCTAGGT (58) |
| 29 | GTCTGTCCAGTGAGGAGATCGG (59) | CATTCTCATCCGGAGGCTAGGT (60) |
| 30 | GTCTGTCCAGTGAGGAGATCGG (61) | CATTCTCATCCGGAGGCTAGGT (62) |
| 31 | GGTCCTGACTTGACCTCGACAG (63) | GAGCACTCAGTCTCGGAAGGG (64) |
| 32 | GGTCCTGACTTGACCTCGACAG (65) | GAGCACTCAGTCTCGGAAGGG (66) |
| 33 | GGTCCTGACTTGACCTCGACAG (67) | GAGCACTCAGTCTCGGAAGGG (68) |
| 34 | GGTCCTGACTTGACCTCGACAG (69) | GAGCACTCAGTCTCGGAAGGG (70) |
| 35 | AGTATGAGCAGGGGCCTCTAGG (71) | CTGGTACCTGCCAGGTCAACTC (72) |
| 36 | GGTGGGGAGTAGACACACCTGA (73) | TCTTCCTCTCCTCCTTTACCTTGC (74) |
| 37 | CATTTCCTAGGTCCTCATCGGTAAA (75) | GAGCAGGTCCTGCAGGTCATAA (76) |
| 38 | CATTTCCTAGGTCCTCATCGGTAAA (77) | GAGCAGGTCCTGCAGGTCATAA (78) |
| 39 | CATTTCCTAGGTCCTCATCGGTAAA (79) | GAGCAGGTCCTGCAGGTCATAA (80) |
| 40 | GAATGTAAGAACATGACCTCCGTGTAG (81) | TGTGTCACCAGGACGGAAGAA (82) |
| 41 | GAATGTAAGAACATGACCTCCGTGTAG (83) | TGTGTCACCAGGACGGAAGAA (84) |
| 42 | CAGACTGCTGCTGGTATTGTGC (85) | AAGGGAGGAAGATCGAATGCC (86) |
| CA-repeats | GGTCAGGATAGATCTCTCAGCT (87) | ACTAATTTCCTCAGAGGCTGTTCAA (88) |

Statistical analysis: The proportion of variation in each SNP attributable to differences between the Japanese and Caucasian populations was estimated using the F_{ST} statistic. Haplotype frequencies for multiple loci were estimated by the expectation-maximization (EM) method using the Arlequin program (Schneider et al. 2000), which is available on the Web at anthropologic unige ch/arlequin.

Pair-wise LD was estimated as D=xᵢⱼ−pᵢpⱼ, where xᵢⱼ is the frequency of haplotype A₁B₁, and p₁ and P₂ are the frequencies of alleles A₁ and B₁ at loci A and B, respectively. A standardized LD coefficient, r, is given by D/(p₁p₂q₁q₂)^{1/2}, where q₁ and q₂ are the frequencies of the other alleles at loci A and B, respectively (Hill and Robertson 1968). Lewontin's coefficient D′ is given by D′, where Dₘₐₓ=min(p₁p₂,q₁q₂) when D<0 or Dₘₐₓ=min(q₁p₂,p₁q₂) when D>0 (Lewontin 1964). Another LD measure for association studies, d², is given by d²=D²/(p₁(1−p₁))², where p₁ is the disease gene frequency. Accordingly, d²=r² p₂(1−p₂)/p₁(1−p₁), where p₂ is the marker allele frequency (Kruglyak 1999).

Evidence of past recombinants in the AGT gene was evaluated using an algorithm that slides a “window” across the DNA sequence and compares the maximum parsimony trees indicated by the two different halves of the window (McGuire and Wright 2000; McGuire et al. 1997). A recombination event is inferred if a discrepancy is supported statistically by a parametric bootstrapping test. This algorithm is implemented in the Topal 2.0 package, available at www.rdg.ac.uk/Statistics/genetics/software.html. Because the tree comparisons require polymorphic variation within the window, a window size of 1500 bp was used. The 12 most common haplotypes were analyzed.

The program ClustalW (Jeanmougin et al. 1998) was used to infer the haplotype tree for common haplotypes observed in Caucasians and Japanese.

### Example 2

### Molecular Variants in AGT

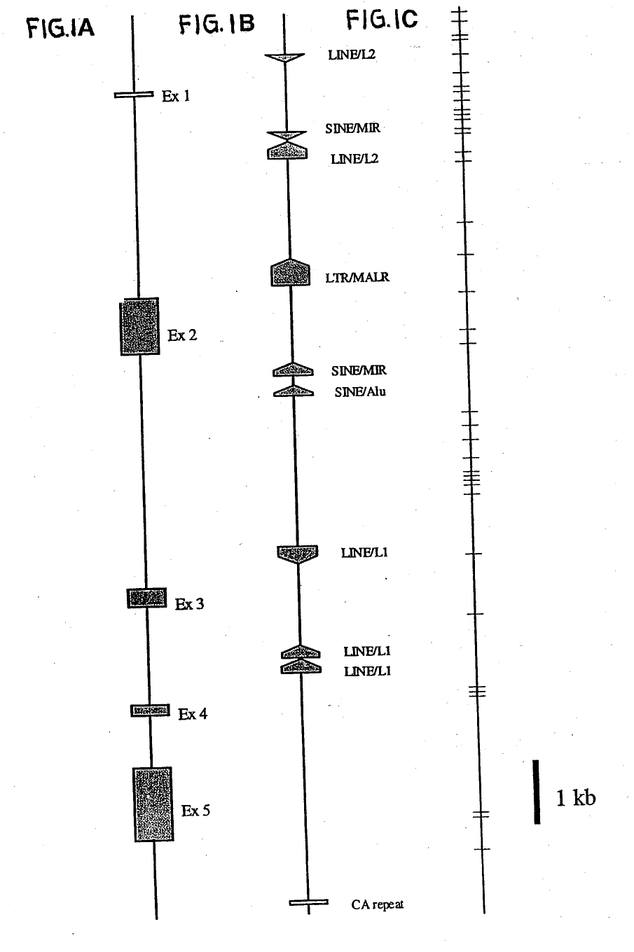

A 14.4 kb genomic region containing the entire AGT gene was completely sequenced. Several known repetitive elements (SINE, LINE, and LTR) and a CA-repeat, the microsatellite used for an early linkage study (Jeunemaitre et al. 1992), were identified (FIG. 1). In total, 44 single nucleotide polymorphisms (SNPs) (one polymorphism per 327 bp) across the scanned sequence were identified in a total of 72 chromosomes from 18. Caucasians and 18 Japanese (FIG. 1C). Among these SNPs, transition substitutions were more prevalent (35 of 44, 79.5%) than transversion substitutions (9 of 44, 20.5%). Forty-one SNPs were found in non-coding regions, and only three were found in coding regions. Other than the CA-repeat, no insertion/deletion polymorphisms were detected.

The 88 Caucasian and 77 Japanese subjects were genotyped for each of the 44 SNPs (Table 2). Forty SNPs were present in both populations, whereas 2 SNPs were present only in Caucasians and 2 SNPs were present only in Japanese. Fifteen SNPs, including A(−6)G and C4072T (the T235M amino acid polymorphism), showed large frequency differences between Caucasians and Japanese (Table 2). The genotype frequencies in the sample fitted Hardy-expectations Weinberg expectations with remarkable fidelity (data not shown). Chimpanzee sequences, which are useful for estimating the ancestral states of SNPs and haplotypes, were determined at the sites corresponding to human SNPs by the direct sequencing of products amplifying the BAC DNA containing the chimpanzee AGT sequence (Table 2).

TABLE 2

Frequency of SNPs in Caucasian and Japanese

| | | | | | |
|---|---|---|---|---|---|
| No. | | | | | |
| of | | Chim- | | | |
| SNP | SNP | panzee | Japanese | Caucasian | F_{ST} |

| | | | | | |
|---|---|---|---|---|---|
| 1 | A-1178G | A | 0.21 | 0.09 | 0.028 |
| 2 | G-1074T | T | 0.21 | 0.09 | 0.028 |
| — | T-829A | T | 0.00 | 0.02 | 0.010 |
| 3 | G-792A | A | 0.21 | 0.09 | 0.028 |
| 4 | T-775C | T | 0.07 | 0.06 | 0.001 |
| 5 | C-532T | C | 0.26 | 0.09 | 0.050 |
| 6 | G-217A | G | 0.21 | 0.09 | 0.028 |
| 7 | A-20C | C | 0.24 | 0.16 | 0.010 |
| 8 | A-6G | A | 0.13 | 0.58 | 0.221 |
| 9 | C67T | C | 0.14 | 0.58 | 0.210 |
| 10 | C172T | C | 0.35 | 0.12 | 0.074 |
| 11 | G384A | G | 0.22 | 0.1 | 0.027 |
| 12 | G400A | G | 0.22 | 0.1 | 0.027 |
| 13 | G507A | G | 0.13 | 0.56 | 0.205 |
| 14 | A676G | G | 0.2 | 0.63 | 0.190 |
| 15 | A698G | G | 0.2 | 0.63 | 0.190 |
| 16 | A1035G | G | 0.41 | 0.72 | 0.098 |
| 17 | A1164G | G | 0.38 | 0.83 | 0.212 |
| 18 | C2079T | C | 0.37 | 0.14 | 0.070 |
| 19 | G2624A | G | 0.33 | 0.1 | 0.078 |
| 20 | A3189G | A | 0.35 | 0.07 | 0.118 |
| 21 | C3889T(T174M) | C | 0.16 | 0.14 | 0.001 |
| — | T3965C(P199P) | T | 0.00 | 0.01 | 0.005 |
| 22 | C4072T(T235M) | C | 0.12 | 0.56 | 0.216 |
| 23 | A5093C | A | 0.13 | 0.55 | 0.197 |
| 24 | C5343T | C | 0.02 | 0.00 | 0.010 |
| 25 | G5556A | G | 0.13 | 0.56 | 0.205 |
| 26 | G5593A | G | 0.13 | 0.56 | 0.205 |
| 27 | A5878C | A | 0.03 | 0.00 | 0.015 |
| 28 | A6066C | C | 0.44 | 0.78 | 0.121 |
| 29 | G6152A | G | 0.25 | 0.09 | 0.045 |
| 30 | C6233T | C | 0.44 | 0.78 | 0.121 |
| 31 | G6309A | G | 0.34 | 0.65 | 0.096 |
| 32 | C6420T | T | 0.34 | 0.2 | 0.025 |
| 33 | C6428G | C | 0.34 | 0.2 | 0.025 |
| 34 | G6442A | G | 0.08 | 0.04 | 0.007 |
| 35 | G7369A | G | 0.32 | 0.12 | 0.058 |
| 36 | C8357T | C | 0.4 | 0.68 | 0.079 |
| 37 | T9597C | T | 0.33 | 0.12 | 0.063 |
| 38 | G9669T | G | 0.33 | 0.12 | 0.063 |
| 39 | A9770G | A | 0.34 | 0.12 | 0.068 |
| 40 | C11535A | C | 0.05 | 0.32 | 0.121 |
| 41 | C11608T | C | 0.05 | 0.33 | 0.127 |
| 42 | C12058A | del | 0.32 | 0.1 | 0.073 |
| Total | | | | | 0.087 |

The extent of nucleotide diversity in each population is shown in Table 3. The average nucleotide diversity, π, is slightly greater in the Japanese sample (9.78±4.88) than in the Caucasian sample (8.36±4.20). The same pattern is seen when θs, the expected proportion of polymorphic sites, is measured. Nucleotide diversity is substantially higher in the 13 kb of noncoding DNA than in 1458 bp of coding sequence. These figures represent slight underestimates because only 72 human chromosomes (36 Japanese and 36 Caucasians) were completely sequenced, with the remainder of the sample genotyped only for the 44 polymorphisms defined in the initial sample. Thus, some rare variants are missed, but this would have only a slight effect on the estimates of π.

TABLE 3
Nucleotide Diversity Values (mean × 10⁻⁴ ± SE × 10⁻⁴)

| | Japanese (n = 154) | | Caucasian (n = 174) | |
|---|---|---|---|---|
| Sequence | π | θ_{S} | π | θ_{S} |
| Coding (1458 bp) | 3.37 ± 3.22 | 2.44 ± 1.82 | 5.19 ± 4.25 | 3.59 ± 2.22 |
| Non-coding | 10.50 ± 5.25 | 5.51 ± 1.53 | 8.72 ± 4.40 | 5.25 ± 1.44 |
| (12,982 bp) | | | | |
| Total (14,400 bp) | 9.78 ± 4.88 | 5.19 ± 1.43 | 8.36 ± 4.20 | 5.08 ± 1.38 |

π is defined as the average proportion of nucleotide differences between
all possible pairs of DNA sequences in the sample. θ_{S} is the expected proportion
of polymorphic sites, given by
$S / {\sum }_{i = 1}^{n - 1} 1 / i ,$
where S is the number of polymorphic sites in the sequence and n
is the number of sequences.

### Example 3

### LDs Between T235M and Other SNPs

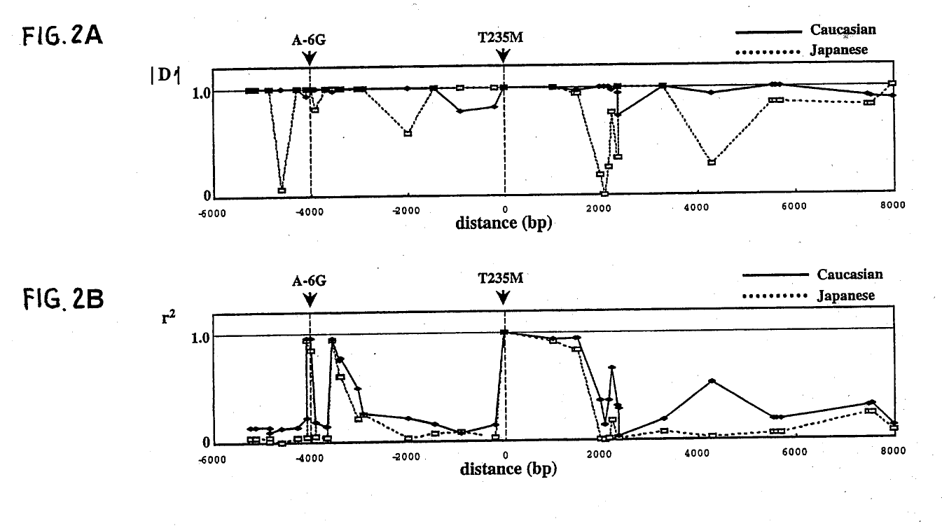

LD between T235M and other SNPs were studied because of the reported association between the T235 allele and EHT. FIG. 2 illustrates substantial differences between D′ and r², in addition to differences between the Japanese and Caucasian samples. The D′ values are generally much higher than the r² values, with a large proportion of D′ values equal to 1.0 or −1.0 (maximum disequilibrium). The percentages of D′ values equal to −1.0 or 1.0 are 53% in the Caucasian sample (412 of 780 total SNP pairs) and 50% in the Japanese sample (427 of 861 SNP pairs). The D′ values equal to 1.0 were caused by the presence of only three of four possible haplotypes for a pair of loci, which forces D to its maximum possible value. When LD was evaluated by r² (FIG. 2A), LD with T235M showed several peaks and valleys and no direct correlation with physical distance. In general, LD values were higher in the Caucasian than in the Japanese sample.

By setting an arbitrary criterion of r²≧0.5, eight SNP alleles (A(−6), C67, G507, A676, A698, A5093, G5556, and G5593) were associated with the T235 allele in both populations (Table 4). The G6309 and C8357 alleles were associated with T235 only in Caucasians. Based on power considerations, Kruglyak (1999) proposed the criterion that d² values>0.1 should be considered “useful” levels of LD. Because r² and d² are almost perfectly correlated in the sample, we designated r²>0.1 as the criterion for useful LD. Table 4 also shows that 35 of 39 (89%) of the SNPs within 7 kb of T235M had an r² value that exceeded 0.1 in the Caucasian population. In the Japanese population, only 33% (13 of 39) of the SNPs met this criterion. As seen in Table 5, highly similar values were seen when disequilibrium between each SNP and the A−6G promoter mutation was evaluated.

TABLE 4
Physical Distance and LD with T235M in Caucasian and Japanese

| | | | | | | | |
|---|---|---|---|---|---|---|---|
| Distance from T235M (kb) | 0-1 | 1-2 | 2-3 | 3-4 | 4-5 | 5-6 | 6-7 |

| | | | | | | | |
|---|---|---|---|---|---|---|---|
| Number of SNPs | 2 | 6 | 7 | 8 | 8 | 5 | 3 |

Caucasian

| | | | | | | | |
|---|---|---|---|---|---|---|---|
| Number of SNPs with r² > 0.1 | 1 | 6 | 6 | 8 | 7 | 5 | 2 |
| (proportion) | (0.50) | (1.00) | (0.86) | (1.00) | (0.88) | (1.00) | (0.67) |
| Number of SNPs with r² > 0.5 | 0 | 3 | 1 | 3 | 3 | 0 | 0 |
| (proportion) | (0.00) | (0.50) | (0.14) | (0.38) | (0.38) | (0.00) | (0.00) |
| mean of r² | 0.102 | 0.588 | 0.29 | 0.45 | 0.39 | 0.159 | 0.24 |
| Japanese | | | | | | | |
| Number of SNPs with r² > 0.1 | 0 | 3 | 2 | 4 | 2 | 0 | 2 |
| (proportion) | (0.00) | (0.50) | (0.29) | (0.50) | (0.25) | (0.00) | (0.67) |
| Number of SNPs with r² > 0.5 | 0 | 3 | 0 | 3 | 2 | 0 | 0 |
| (proportion) | (0.00) | (0.50) | (0.00) | (0.38) | (0.25) | (0.00) | (0.00) |
| mean of r² | 0.052 | 0.448 | 0.065 | 0.317 | 0.243 | 0.046 | 0.173 |

TABLE 5
Physical Distance and LD with A-6G in Caucasian and Japanese

| | | | | | | | | | | | |
|---|---|---|---|---|---|---|---|---|---|---|---|
| Distance from A-6G (kb) | 0-1 | 1-2 | 2-3 | 3-4 | 4-5 | 5-6 | 6-7 | 7-8 | 8-9 | 9-10 | >10 |
| Number of SNPs | 12 | 4 | 2 | 2 | 1 | 3 | 7 | 1 | 1 | 3 | 3 |

Caucasian

| | | | | | | | | | | | |
|---|---|---|---|---|---|---|---|---|---|---|---|
| Number of SNPs with r² > 0.1 | 11 | 4 | 2 | 1 | 1 | 3 | 6 | 1 | 1 | 3 | 3 |
| (proportion) | (0.92) | (1.00) | (1.00) | (0.50) | (1.00) | (1.00) | (0.86) | (1.00) | (1.00) | (1.00) | (1.00) |
| Number of SNPs with r² > 0.5 | 4 | 0 | 1 | 0 | 1 | 3 | 1 | 0 | 0 | 0 | 0 |
| (proportion) | (0.33) | (0.00) | (0.50) | (0.00) | (1.00) | (1.00) | (0.14) | (0.00) | (0.00) | (0.00) | (0.00) |
| mean of r² | 0.386 | 0.248 | 0.43 | 0.106 | 0.96 | 0.902 | 0.308 | 0.186 | 0.477 | 0.186 | 0.231 |
| Japanese | | | | | | | | | | | |
| Number of SNPs with r² > 0.1 | 4 | 2 | 0 | 0 | 1 | 3 | 1 | 0 | 0 | 0 | 2 |
| (proportion) | (0.33) | (0.50) | (0.00) | (0.00) | (1.00) | (1.00) | (0.14) | (0.00) | (0.00) | (0.00) | (0.67) |
| Number of SNPs with r² > 0.5 | 4 | 0 | 0 | 0 | 1 | 3 | 0 | 0 | 0 | 0 | 0 |
| (proportion) | (0.33) | (0.00) | (0.00) | (0.00) | (1.00) | (1.00) | (0.00) | (0.00) | (0.00) | (0.00) | (0.00) |
| mean of r² | 0.291 | 0.134 | 0.062 | 0.056 | 0.94 | 0.922 | 0.045 | 0.07 | 0.023 | 0.055 | 0.165 |

The results demonstrate that significant LD is found between putative susceptibility alleles in the A GT region and other SNPs. However, the pattern of LD in this region is highly irregular, with some pairs of closely linked SNPs showing little LD. This irregularity has been observed in many previous studies of small genomic regions (Abecasis et al. 2001; Jorde 1995; Jorde et al. 1994; Jorde et al. 1993; MacDonald et al. 1991; Nickerson et al. 1998; Taillon-Miller et al. 2000) and is to be expected because recombination becomes rare relative to other events that can affect LD, such as mutation and gene conversion. The results show evidence of only a few historical recombinants in this region. This paucity of recombinants helps to explain why D′ values are at 1.0 for many pairs of polymorphisms: recombination is more likely to generate two new haplotypes from two polymorphic sites, giving rise to a total of four haplotypes. On the other hand, if a new haplotype is generated by mutation, a total of three haplotypes is likely to be seen, and D′ for two sites will equal 1.0. The result is that D′ is a relatively insensitive measure of LD in this small genomic region.

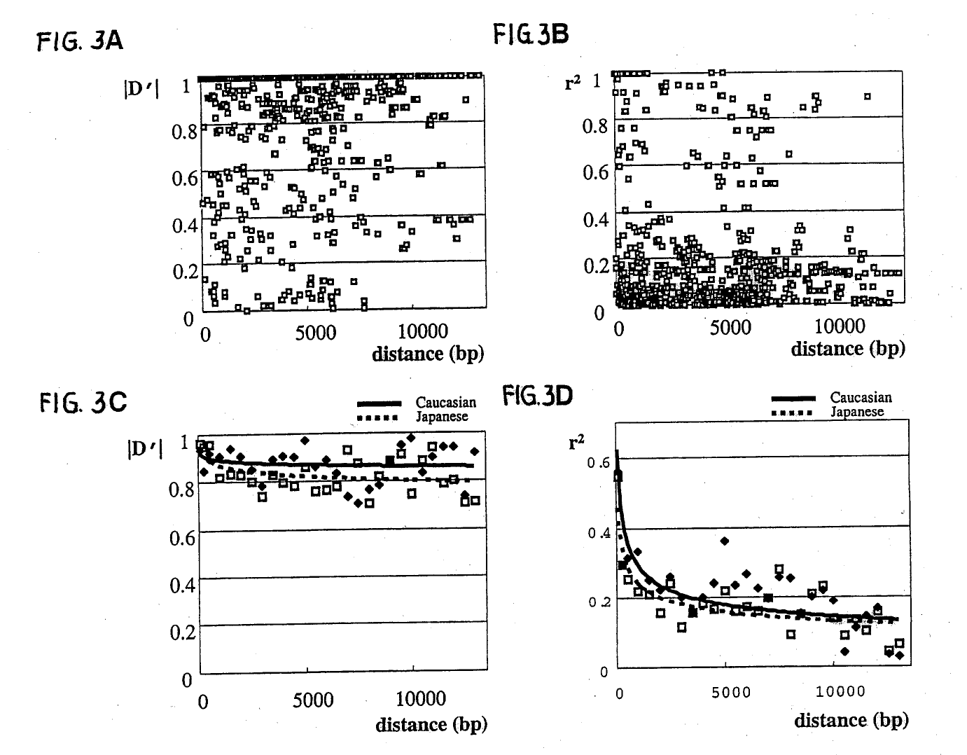

We observed a slightly more regular pattern of LD decline with physical distance when LD values were averaged across 500-bp intervals (FIG. 3). This procedure is expected to smooth out some of the variation in LD estimates, and similar results have been obtained in other studies in which LD values are averaged across genomic intervals (Abecasis et al. 2001; Dunning et al. 2000).

### Example 4

### Pair-Wise LD in AGT

When all the possible pair-wise LDs in Japanese individuals, evaluated by D′ or r², were plotted as a function of physical distance, LD did not decline smoothly with increasing distance between SNPs (FIGS. 3A and 3B). However, the average values of D′ (FIG. 3C) and r² (FIG. 3D) in each 500 bp interval declined markedly with physical distance. For both measures, the Caucasian sample showed a higher level of LD than did the Japanese sample.

The d² statistic for each pair of SNPs was measured assuming that the SNP containing the least common minor allele was the disease-causing variant. As expected from the mathematical similarity between d² and r², the pairwise values of these two measures were highly correlated (Pearson's r=0.96). The correlation between d² and D′ was much lower (Pearson's r=0.33), reflecting the large number of D′ values equal to 1.0 or −1.0.

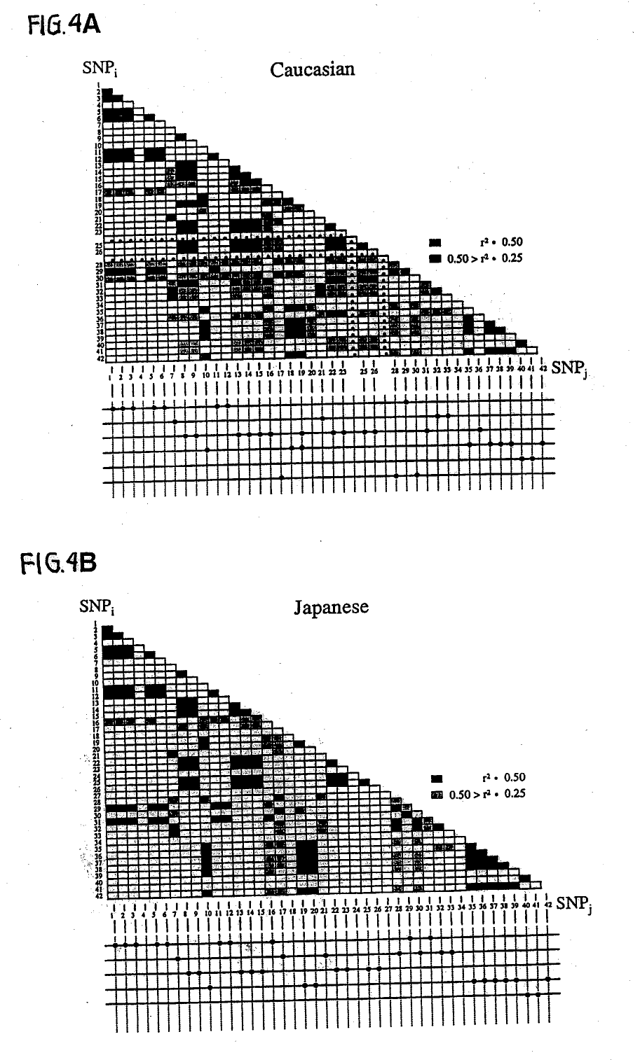

To assess patterns of significant disequilibrium values in the two populations, FIG. 4 shows pairwise r² values exceeding 0.5 (black) and ranging between 0.25 and 0.5 (gray). The value r²=0.5 is equivalent to χ2=88 (p<10⁻¹⁹) in 176 Caucasian chromosomes and χ²=77 (p<10⁻¹⁷) in 154 Japanese chromosomes. The distribution of LD is highly similar in the two populations, and at least 5 major SNP subgroups with minor changes were present (bottom of FIG. 4).

Although the average LD values decline with physical distance, some pairs of SNPs exhibit significant LD at distances of nearly 10 kb. This is consistent with the results of many other empirical studies, some of which detect significant LD at distances up to several hundred kb (Ajioka et al. 1997; Huttley et al. 1999; Jorde et al. 1994; Jorde et al. 1993; Lonjou et al. 1999; Moffatt et al. 2000; Peterson et al. 1995; Reich et al. 2001; Stephens et al. 2001). These empirical results stand in contrast to a simulation study that predicted little or no useful LD beyond distances of 10 kb (Kruglyak 1999). This study assumed either constant population size or simple exponential growth, both of which are likely to be over-simplifications (Wall and Przeworski 2000). Cyclic bottlenecks and expansions, for example, can lead to higher LD levels (Collins et al. 1999). In addition, the simulation study ignored the potential effects of natural selection on disease-causing variants. Natural selection limits the length of time during, which these variants can persist in populations, reducing the length of time during which LD can dissipate (Terwilliger and Weiss 1998). These and other factors are likely to account for discrepancies between these simulation results and the empirical studies reported thus far.

Comparisons of LD patterns in the Japanese and Caucasian populations showed that, while the overall patterns were quite similar, there was substantially greater LD in the Caucasian sample. In particular, 89% of the SNPs within 7 kb of the EHT-associated T235M polymorphism demonstrated “useful” LD (r²>0.1) in the Caucasian sample, but this figure was only 33% in the Japanese sample. Thus, the probability of detecting the EHT-associated polymorphism in a genome LD scan would be substantially greater in the Caucasian population. The higher level of LD in this Utah CEPH sample may reflect the substantial genetic homogeneity that has been demonstrated in genetic studies of this population (McLellan et al. 1984; O'Brien et al. 1994; O'Brien et al. 1996). Other studies have also demonstrated substantial differences in LD in various populations (Kidd et al. 1998; Reich et al. 2001; Tishkoff et al. 1996; Tishkoff et al. 1998; Tishkoff et al. 2000), highlighting the effects of population history on LD patterns.

### Example 5

### Haplotype Analysis

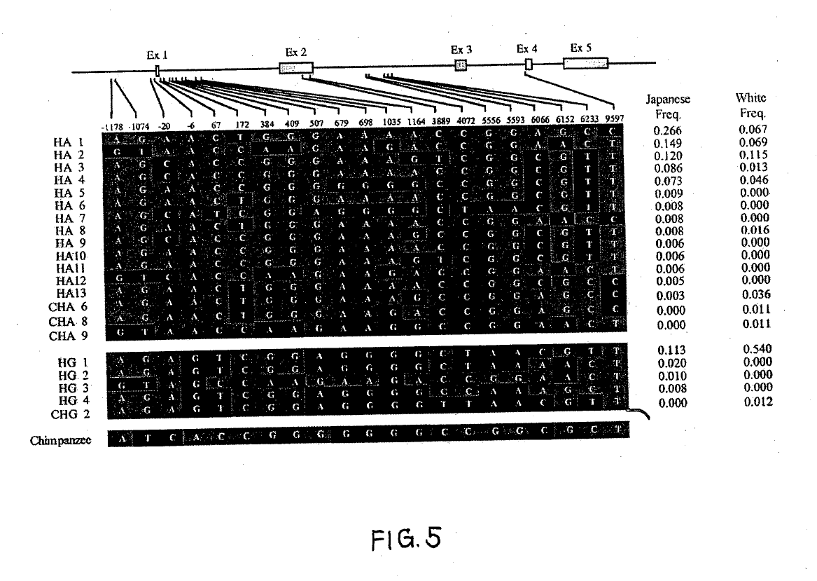

Haplotypes were constructed based on the genotype data from 21 SNPs selected to span most of the AGT gene. Haplotype frequencies were estimated using the EM algorithm with phase-unknown samples. This procedure has been shown to estimate common haplotype frequencies accurately when the Hardy-Weinberg assumption is fulfilled and when sample sizes are reasonably large (e.g., >100 chromosomes) (Fallin and Schork 2000; Tishkoff et al. 2000). Accordingly, the Japanese sample was expanded to 188 unrelated individuals for this analysis. The haplotypes carrying A(−6) and T235 could be subdivided into five major haplotypes, HA1, HA2, HA3, HA4, and HA5. Only one major haplotype carrying G(−6) and M235, the HG1 haplotype, was present in both populations. FIG. 5 shows the haplotypes that were estimated to be present in 2 or more copies in at least one of the populations. Caucasians and Japanese shared the six frequent haplotypes, even though the frequencies of those haplotypes were quite different between the two populations. In Caucasians, the HG1 haplotype, which is thought to be protective for EHT, had a frequency of 54%. Haplotype diversity, (2n(1−●xᵢ²)/(2n−1), where xᵢ is the frequency of haplotype i and n is sample number, was estimated as 0.684 for the Caucasians and 0.872 for the Japanese.

### Example 6

### Recombination Analysis

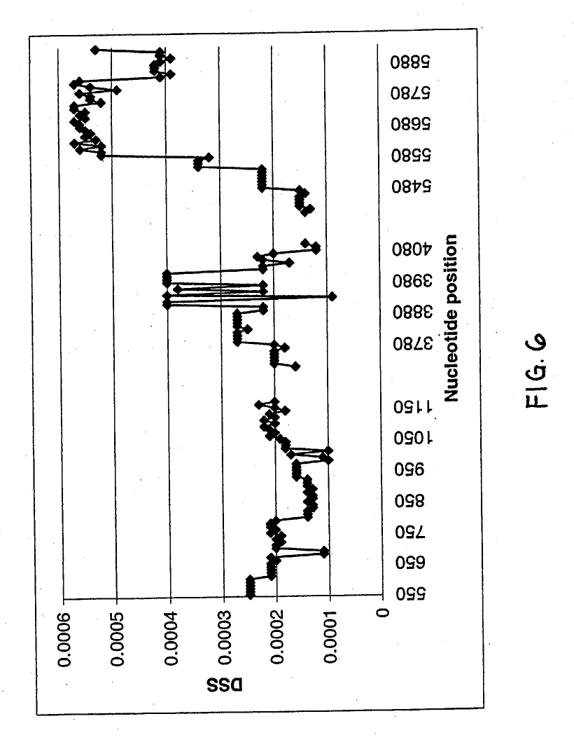

Evidence of past recombinants in the AGT sequence is given by the DSS (difference in sum of squares) values plotted in FIG. 6 (y axis) against position in the AGT sequence (x axis). Higher DSS values indicate greater discrepancies between the two trees generated by each half of the sliding window of DNA sequence and thus reflect the likely locations of recombinants. FIG. 6 provides evidence for recombinant events at approximately positions 550, 3800, 5600, and 6000 (possible recombinants upstream and downstream of these locations could not be discerned because of the locations of polymorphisms and limitations on the window size). The bootstrap analysis showed that the DSS values at each of these positions differed significantly from zero. These inferred recombinants correspond to blocks of SNPs that are in association with one another, as seen in FIGS. 4 and 5. One block begins with SNP 13 (G507A) and ends with SNP 17 (Al 164G). A second block begins with SNP 22 (the T235M polymorphism, C4072T) and ends with SNP 28 (A6066C).

### Example 7

### Gene Tree for Common Haplotypes Observed in Japanese and Caucasians

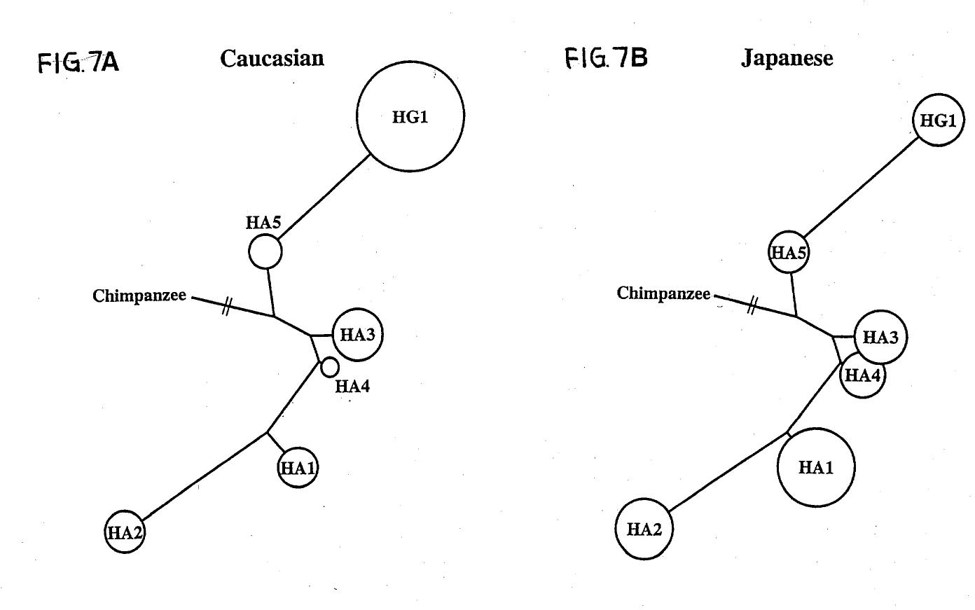

A haplotype tree for the major haplotypes was constructed using the ClustalW program (FIG. 7). Chimpanzee sequences were used to determine the ancestral haplotype. The HG1 and HA1 haplotypes, the most frequent haplotypes for Caucasians and Japanese, respectively, are remotely related to the chimpanzee sequence.

### Example 8

### Relationship Between SNP Haplotypes and Microsatellite Marker

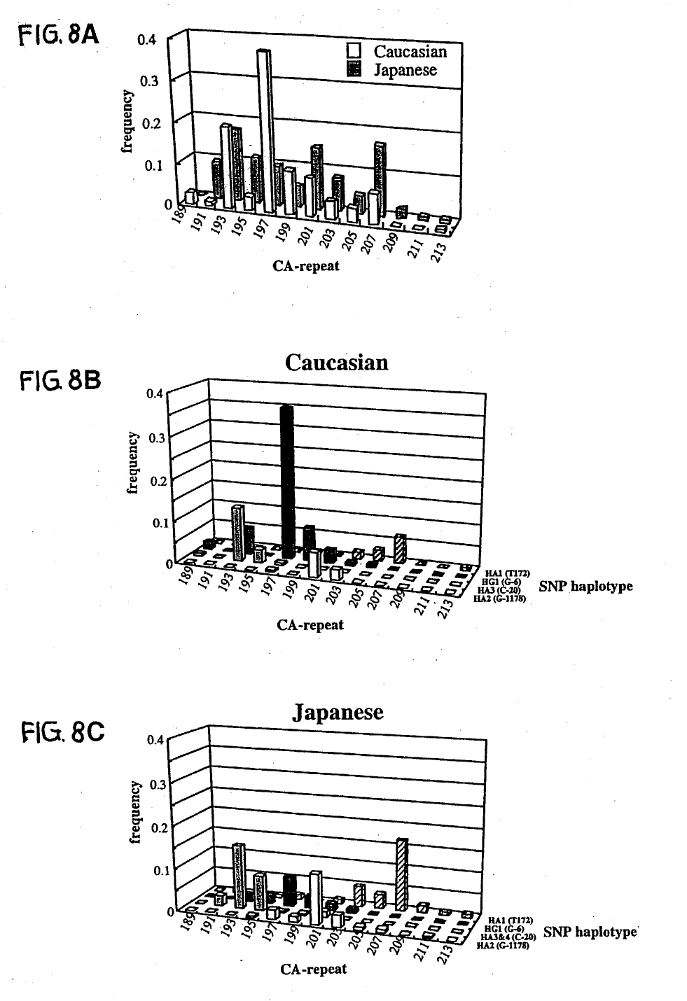

The CA-repeat, which is located downstream of exon 5, was identified previously (Katelevtsev et al. 1991) and was used for linkage studies. The relationship between the four most common SNP haplotypes and the microsatellite alleles is shown in FIG. 8. Although the distribution of CA-repeat alleles varies between Caucasians and Japanese, the association patterns between each SNP haplotype and the microsatellite alleles are very similar in the two populations. The same microsatellite allele is in association with each SNP haplotype in both populations (e.g., microsatellite allele 197 and the HG1 haplotype).

The notable successes of LD in localizing genes responsible for Mendelian disorders (Feder et al. 1996; Hästbacka et al. 1994), combined with the availability of hundreds of thousands of SNPs throughout the genome (Sachidanandam et al. 2001), has sparked a strong interest in the use of LD methods for localizing genes underlying complex diseases (Collins et al. 1997; Jorde 2000; Jorde et al. 2001; Kruglyak 1999; Pritchard and Przeworski 2001; Reich et al. 2001; Risch and Merikangas 1996; Risch 2000; Schork et al. 2001; Stephens et al. 2001). Many important questions regarding this approach remain unanswered, however. For example, the following remain unknown issues: to what extent LD are patterns affected by factors such as chromosome location, isochore structure, and choice of markers; how evolutionary factors, including natural selection, gene flow, genetic drift, population subdivision, and gene conversion, affect LD; and which types of populations are best suited to LD mapping. Answers to these questions are necessary for the efficient design of LD studies.

Variation in AGT has been shown to correlate with variation in plasma angiotensinogen and with risk of hypertension. Therefore, this gene provides the basis for a useful case study of LD patterns in a locus that helps to determine susceptibility to a complex disease. The results demonstrate that significant LD is found between putative susceptibility alleles in the AGT region and other SNPs. However, the pattern of LD in this region is highly irregular, with some pairs of closely linked SNPs showing little LD. This irregularity has been observed in many previous studies of small genomic regions (Abecasis et al. 2001; Jorde 1995; Jorde et al. 1994; Jorde et al. 1993; MacDonald et al. 1991; Nickerson et al. 1998; Taillon-Miller et al. 2000) and is to be expected because recombination becomes tare relative to other events that can affect LD, such as mutation and gene conversion. The results show evidence of only a few historical recombinants in this region. This paucity of recombinants helps to explain why D′ values are at 1.0 for many pairs of polymorphisms: recombination is more likely to generate two new haplotypes from two polymorphic sites, giving rise to a total of four haplotypes. On the other hand, if a new haplotype is generated by mutation, a total of three haplotypes is likely to be seen, and D′ for two sites will equal 1.0. The result is that D′ is a relatively insensitive measure of LD in this small genomic region.

A slightly more regular pattern of LD decline with physical distance was observed when LD values were averaged across 500-bp intervals (FIG. 3). This procedure is expected to smooth out some of the variation in LD estimates, and similar results have been obtained in other studies in which LD values are averaged across genomic intervals (Abecasis et al. 2001; Dunning et al. 2000).

Although the average LD values decline with physical distance, some pairs of SNPs exhibit significant LD at distances of nearly 10 kb. This is consistent with the results of many other empirical studies, some of which detect significant LD at distances up to several hundred kb (Ajioka et al. 1997; Huttley et al. 1999; Jorde et al. 1994; Jorde et al. 1993; Lonjou et al. 1999; Moffatt et al. 2000; Peterson et al. 1995; Reich et al. 2001; Stephens et al. 2001). These empirical results stand in contrast to a simulation study that predicted little or no useful LD beyond distances of 10 kb (Kruglyak 1999). This study assumed either constant population size or simple exponential growth, both of which are likely to be over-simplifications (Wall and Przeworski 2000). Cyclic bottlenecks and expansions, for example, can lead to higher LD levels (Collins et al. 1999). In addition, the simulation study ignored the potential effects of natural selection on disease-causing variants. Natural selection limits the length of time during which these variants can persist in populations, reducing the length of time during which LD can dissipate (Terwilliger and Weiss 1998). These and other factors are likely to account for discrepancies between these simulation results and the empirical studies reported thus far.

Comparisons of LD patterns in the Japanese and Caucasian populations showed that, while the overall patterns were quite similar, there was substantially greater LD in the Caucasian sample. In particular, 89% of the SNPs within 7 kb of the EHT-associated T235M polymorphism demonstrated “useful” LD (r²>0.1) in the Caucasian sample, but this figure was only 33% in the Japanese sample. Thus, the probability of detecting the EHT-associated polymorphism in a genome LD scan would be substantially greater in the Caucasian population. The higher level of LD in this Utah CEPH sample may reflect the substantial genetic homogeneity that has been demonstrated in genetic studies of this population (McLellan et al. 1984; O'Brien et al. 1994; O'Brien et al. 1996). Other studies have also demonstrated substantial differences in LD in various populations (Kidd et al. 1998; Reich et al. 2001; Tishkoff et al. 1996; Tishkoff et al. 1998; Tishkoff et al. 2000), highlighting the effects of population history on LD patterns.

It is instructive to compare haplotype complexity in AGT with that of the lipoprotein lipase (LPL) gene. The AGT region, with an average nucleotide diversity value (π) of approximately 1/1,000, is typical of most regions reported thus far (Jorde et al. 2001; Sachidanandam et al. 2001; Wall and Przeworski 2000). The LPL gene has a somewhat higher level of nucleotide diversity (π=1/500) and exhibits a high degree of haplotype complexity in several different populations, with evidence of multiple recombinant events (Clark et al. 1998; Nickerson et al. 1998; Templeton et al. 2000). Indeed, haplotype reconstruction showed that, for most (64%) pairs of SNPs in the LPL region, all four haplotypes were present. In contrast, most pairs of SNPs in the AGT region yielded evidence of only three haplotypes (50% in the Japanese sample and 53% in the Caucasian sample), indicating less recombination. Just six leading haplotypes (FIG. 5) account for 84% of the 176 Caucasian chromosomes and 73% of the 376 Japanese chromosomes. Thus, relatively few SNPs can account for much of the variation in the AGT region, implying that this gene would require a lower SNP density for association detection than would a more complex gene like LPL.

Taken together, these results demonstrate that it is not feasible to predict a uniform SNP density for genome-wide association studies. The density of SNPs needed to detect disease-associated polymorphisms will vary with genomic region, marker type, and choice of population. In addition, the distribution of LD is almost guaranteed to be irregular in relatively small genomic regions, particularly in more recently founded populations that have a relatively brief history of recombination. More empirical information is needed about the effects of all of these factors on LD patterns in order to design efficient association studies.

The haplotype patterns seen in the Japanese and Caucasian populations allow some inferences about the history of the EHT-associated AGT polymorphisms. As seen in FIG. 4, LD and haplotype patterns are quite similar in the two populations, and both share the same major haplotypes (albeit with different frequencies). In addition, the same CA-repeat alleles are found in association with each major haplotype in the two populations. In particular, the M235 allele occurs on the same haplotype background, and this haplotype is quite common in two populations of distinct geographic origin (Japan versus the northern European origin of the Utah population). These results, taken together with the fact that the T235M polymorphism is seen in at least some African populations (Corvol and Jeunemaitre 1997), indicate that the polymorphism probably arose before modem humans left Africa and was shared by a portion of the population that eventually populated Europe and Asia. Predating the African exodus, the polymorphism is likely to be at least 50,000 years old (Hedges 2000; Jorde et al. 1998; Underhill et al. 2000).

The results also bear on the question of natural selection for variation in the AGT gene. Notably, the highest F_{ST} values seen in Table 2 are those associated with the A−6G promoter variant and the T235M polymorphism, both of which are associated with hypertension. Exceptionally high F_{ST} values are a potential indication of the effects of directional selection (Beaumont and Nichols 1999; Bowcock et al. 1991; Lewontin and Krakauer 1973). An analysis of several nonhuman primate species (chimpanzee, gorilla, orangutan, gibbon, baboon, and macaque) shows that the T235 allele is fixed in these species (Dufour et al. 2000; Inoue et al. 1997). In addition, the A(−6) promoter variant is fixed in the three species examined thus far (chimp, gorilla, and macaque). Thus, the protective M235 and G(−6) variants are likely to have arisen during the course of human evolution. The T235 allele varies widely in frequency: approximately 35-45% in Caucasians, 75-80% in Asians, 75-80% in African-Americans, and 90% or more in Africans (Corvol and Jeunemaitre 1997; Staessen et al. 1999). This pattern leads to the hypothesis that the A(−6)/T235 haplotype, associated with higher angiotensinogen expression and greater sodium reabsorption, was adaptive in the tropical, sodium-poor environment of sub-Saharan Africa (Jeunemaitre et al. 1997) but was selected against (or became selectively neutral) as modem humans radiated out of Africa into other environments. Signatures of natural selection (Kreitman 2000) in the AGT gene should be evaluated in multiple populations to test this intriguing hypothesis.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### BIBLIOGRAPHY

Abecasis G R, et al. (2001). Am J Hum Genet 68:191-197.

Ajioka R S, et al. (1997). Am J Hum Genet 60:1439-1447

Beaumont M A, et al. (1999). Proc R Soc Lond B 263:1619-1626

Bengtsson K, et al. (1999). J Hypertens 17:1569-75.

Bishop, D. T. and Williamson, J. A. (1990). Am. J Hum. Genet. 46:254-265.

Blackwelder, W. C. and Elston, R. C. (1985). Genet. Epidemiol. 2:85-97.

Bonnen P E et al. (2000). Am J Hum Genet 67:1437-51.

Borman S (1996). Chemical & Engineering News, December 9 issue, pp. 42-43.

Bowcock A M, et al. (1991). Proc Natl Acad Sci USA 88:839-843

Brand E, et al. (1998). Hypertension 31:725-9.

Campbell, D. J., and Habener, J. F. (1986). J Clin. Invest. 78:1427-1431.

Chee M, et al. (1996). Science 274:610-614.

Clauser, E., et al. (1989). Am. J. Hypertens. 2:403-410.

Collins A, et al. (1999). Proc Natl Acad Sci USA 96:15173-15177

Collins F S, et al. (1997). Science 278:1580-1581

Corvol P, et al. (1997). Endocr Rev 18:662-77

Corvol P, et al. (1999). Hypertension 33:1324-31.

DeRisi J, et al. (1996). Nat. Genet. 14:457-460.

Dufour C, et al. (2000). Genomics 69:14-26.

Dunning A M, et al. (2000). Am J Hum Genet 67:1544-54

Eaton, S. B., et al. (1985). N. Engl. J Med. 312:283-289.

Fallin D, et al. (2000). Am J Hum Genet 67:947-59

Feder J N, et al. (1996). Nature Genet 13:399-408

Fodor, S. P. A. (1997). DNA Sequencing. Massively Parallel Genomics. Science 277:393-395.

Fukamizu, A., et al. (1989). J Biol. Chem. 265:7576-7582.

Gaillard, I., et al. (1989). DNA 8:87-99.

Gardes, J., et al. (1982). Hypertension 4:185-189.

Grompe, M., (1993). Nature Genetics 5:111-117.

Grompe, M., et al., (1989). Proc. Natl. Acad. Sci. USA 86:5855-5892.

Hacia J G, et al. (1996). Nature Genetics 14:441-447.

Hall, J. E., and Guyton, A. C. (1990). In: Hypertension: Pathophysiology Diagnosis and Management, Laragh, J. H. and Brenner, B. M., eds., (Raven Press, Ltd., N.Y.), pp. 1105-1129.

Harrop, S. H., et al. (1990). Hypertension 16:603-614.

Hästbacka J, et al. (1994). Cell 78:1073-1087

Hedges S B (2000). Nature 408:652-3.

Hilbert, P., et al. (1991). Nature 353:521-528.

Hill W G, et al. (1968). Theor Appl Genet 38:226-231

Huttley G A., et al. (1999). Genetics 152:1711-1722

Inoue I, et al. (1997). J Clin Invest 99:1786-97.

Iso H, et al. (2000). J Hypertens 18:1197-206.

Jacob, H. J., et al. (1991). Cell 67:213-224.

Jeanmougin F, et al. (1998). Trends Biochem Sci 23:403-5.

Jeunemaitre, X., et al. (1992a). Nature Genetics 1:72 75.

Jeunemaitre, X., et al. (1992b). Hum. Genet. 88:301-306.

Jeunemaitre, X., et al. (1992c). Cell 71:169-178.

Jeunemaitre, X., et al. (1997). Am J Hum. Genet. 60:1448-1460.

Joint National Committee on Detection, Evaluation and Treatment of Hypertension (1985). Final report of the Subcommittee on Definition and Prevalence Hypertension 7:457-468.

Jorde L B (1995). Am J Hum Genet 56:11-14

Jorde L B (2000). Genome Res 10:1435-44

Jorde L B, et al. (1998). Bio Essays 20:126-136

Jorde L B, et al. J (2001). Hum Molec Genet (in press)

Jorde L B, et al. (1994). Am J Hum Genet 54:884-898

Jorde L B, et al. (1993). Am J Hum Genet 53:1038-1050

Kato N, et al. (1999). J Hypertens 17:757-63.

Kidd J R, et al. (2000). Am J Hum Genet 66:1882-1899

Kidd K K, et al. (1998). Hum Genet 103:211-227

Kinszler, K. W., et al. (1991). Science 251:1366-1370.

Kreitman M (2000). Annu Rev Genomics Hum Genet 1:539-559

Kruglyak L (1999). Nature Genet 22:139-144

Kunz R, et al. (1997). Hypertension 30:1331-7.

Kurtz, T. W., et al. (1990). J. Clin. Invest. 85:1328-1332.

Laan M, et al. (1997). Nature Genet 17:435-438

Lalouel J M (2001). Adv Genet 42:517-33.

Lalouel, J. M. (1990). In: Drugs Affecting Lipid Metabolism, A. M. Gotto and L. C. Smith (eds.), Elsevier Science Publishers, Amsterdam, pp. 11-21.

Lander, E. S., and Botstein, D. (1986). Cold Spring Harbor Symp. Quant. Biol. 51:46-61.

Lander, E. S., and Botstein, D. (1989). Genetics 121:185 199.

Larson N, et al. (2000). Hypertension 35:1297-300.

Lathrop, G. M., and Lalouel, J. M. (1991). In: Handbook of Statistics, Vol. 8 (Elsevier Science Publishers, Amsterdam), pp. 81-123.

Lathrop, G. M., et al. (1984). Proc. Natl. Acad. Sci. USA 81:8443-3446.

Lewontin R C (1964). Genetics 49:49-67

Lewontin R C, et al. (1973). Genetics 74:175-195

Lipshutz R J, et al. (1995). BioTechniques 19:442-447.

Lockhart D J, et al. (1996). Nature Biotechnology 14:1675-1680.

Lonjou C, et al. (1999). Proc Natl Acad Sci USA 96:1621-1626

MacDonald M E, et al. (1991). Am J Hum Genet 49:723-734

McGuire G, et al. (2000). Bioinformatics 16:130-134

McGuire G, et al. (1997). Molec Biol Evol 14:1125-1131

McLellan T, et al. (1984). Am J Hum Genet 36:836-857

Menard, J., and Catt, K. J. (1973). Endocrinology 92:1382-1388.

Menard, J., et al. (1991). Hypertension 18:705-706.

Moffatt M F, et al. (2000). Hum Mol Genet 9:1011-9.

Mullins, J. J., et al. (1990). Nature 34:541-544.

Nakajima T., et al. (2000). J Hum Genet 45:212-7.

Nakajima T., et al. (2002). Am J Hum Genet 70(1):108-23.

Nickerson D A, et al. (1998). Nature Genet 19:233-240

Niu T, et al. (1999). Ann Epidemiol 9:245-53.

O'Brien E et al. (1994). Hum Biol 66:743-759

O'Brien E, et al. (1996). Am J Hum Biol 8:609-614

Ohkubo, H., et al. (1990). Proc. Nat. Acad. Sci. USA 87:5153-5157.

Pan W H, et al. (2000). Hum Genet 107:210-5.

Peterson A C et al. (1995). Hum Molec Genet 4:887-894

Pritchard J K, et al. (2001). Am J Hum Genet 69:1-14.

Province M A, et al. (2000). J Hypertens 18:867-76.

Rankinen T, et al. (2000). Am J Physiol Heart Circ Physiol 279:H368-74.

Rapp, J. P., et al. (1989). Science 243:542-544.

Reich D E, et al. (2001). Nature 411:199-204.

Rice T, et al. (2000). Circulation 102:1956-63.

Risch N, et al. (1996). Science 273:1516-1517

Risch N J (2000). Science 405:847-856

Sachidanandam R, et al. (2001). Nature 409:928-33.

Sassaho, P., et al. (1987). Am. J Med. 83:227-235.

Sato N, et al. (2000). Life Sci 68:259-72.

Schneider S, et al. (2000) Arlequin: a software for population genetic data analysis. University of Geneva, Geneva

Schork N J, et al. (2001). Adv Genet 42:191-212.

Sealey, J. E., and Laragh, J. H. (1990). In: Hypertension: Pathophysiology. Diagnosis and Management, J. H. Laragh and B. M. Brenner, eds. (Raven Press, New York), pp. 1287-1317.

Sheffield, V. C., et al. (1989). Proc. Natl. Acad. Sci. USA 86:232-236.

Sheffield, V. C., et al. (1991). Am. J. Hum. Genet. 49:699-706.

Shoemaker D D, et al. (1996). Nature Genetics 14:450-456.

Staessen J A, et al. (1999). J Hypertens 17:9-17.

Stephens J C, et al. (2001). Science 293:489-493

Suarez, B. K., et al. (1978). Ann. Hum. Genet. 42:87-94.

Suarez, B. k. et al. (1983). Ann. Hum. Genet. 47:153-159.

Suarez, B. K., and Van Eerdewegh, P. (1984). Am. J Med. Genet. 18:135 146.

Taillon-Miller P, et al. (2000). Nat Genet 25:324-8.

Taittonen L, et al. (1999). Am J Hypertens 12:858-66.

Templeton A R, et al. (2000). Am J Hum Genet 66:69-83.

Terwilliger J D, et al. (1998) Curr Opin Biotechnol 9:578-94

Tishkoff SA, et al. (1996). Science 271:1380-1387

Tishkoff S A, et al. (1998). Am J Hum Genet 62:1389-1402

Tishkoff S A, et al. (2000). Am J Hum Genet 67:518-22

Tishkoff S A, et al. (2000). Am J Hum Genet 67:901-25

Underhill P A, et al. (2000). Nat Genet 26:358-61

Walker, W. G., et al. (1979). Hypertension 1:287 291.

Wall J D, et al. (2000) Genetics 155:1865-1874

Ward, R. (1990). In: Hypertension: Pathophysiology. Diagnosis and Management, Laragh, J. H. and Brenner, B. M., eds., (Raven Press, Ltd., New York), pp. 81-100.

White, M. B., et al., (1992). Genomics 12:301-306.

Xiong M, et al. (1998). Hum Hered 48:295-312

Yu A, et al. (2001). Nature 409:951-3.

Zavattari P, et al. (2000). Hum Mol Genet 9:2947-57

Watt, G. C. M., et al. (1992). J Hypertens. 10:473-482.

White, R. L., and Lalouel, J. M. (1987). In: Advances in Human Genetics, Vol. 16, H. Harris and K. Hirschhorn, eds. (Plenum Press, New York), pp. 121-228.

What is claimed is:

1. A method for determining the predisposition of an individual to hypertension which comprises analyzing at least part of the DNA sequence of the angiotensinogen (AGT) gene of said individual for the presence of at least one single nucleotide polymorphism (SNP) in the A GT gene, wherein said SNP is selected from the group consisting of:

| | |
|---|---|
| (a) | A-1178G; |
| (b) | G-1074T; |
| (c) | T-829A; |
| (d) | G-792A; |
| (e) | T-775C; |
| (f) | C-532T; |
| (g) | G-217A; |
| (h) | C172T; |
| (i) | G384A; |
| (j) | G400A; |
| (k) | G507A; |
| (l) | A676G; |
| (m) | A698G; |
| (n) | A1035G; |
| (o) | A1164G; |
| (p) | C2079T; |
| (q) | G2624A; |
| (r) | A3189G; |
| (s) | T3965C(P199P); |
| (t) | A5093C; |
| (u) | C5343T; |
| (v) | G5556A; |
| (w) | G5593A; |
| (x) | A5878C; |
| (y) | A6066C; |
| (z) | G6152A; |
| (aa) | C6233T; |
| (ab) | G6309A; |
| (ac) | C6420T; |
| (ad) | C6428G; |
| (ae) | G6442A; |
| (af) | G7369A; |
| (ag) | C8357T; |
| (ah) | T9597C; |
| (ai) | G9669T; |
| (aj) | A9770G; |
| (ak) | C11535A; |
| (al) | C11608T; and |
| (am) | G12058A. |

2. The method of claim 1 wherein said predisposition is a predisposition to essential hypertension.

3. The method of claim 1 wherein said predisposition is a predisposition to pregnancy-induced hypertension.

4. The method of claim 1 wherein the genomic sequence of the AGT gene of said individual is analyzed.

5. The method of claim 1 wherein the genomic sequence of a part of the AGT gene of said individual is analyzed.

6. The method of claim 1 wherein said determination of at least a part of the AGT gene is performed by hybridization of a nucleic acid to the AGT gene of said individual.

7. The method of claim 6 wherein said hybridization is performed with an allele-specific oligonucleotide probe.

8. The method of claim 1 wherein said analysis is carried out by sequence analysis.

9. The method of claim 1 wherein said determination of the AGT gene is carried out by SSCP analysis.

10. A nucleic acid probe which specifically hybridizes to an SNP in the AGT gene wherein said SNP is selected from the group consisting of:

| | |
|---|---|
| (a) | A-1178G; |
| (b) | G-1074T; |
| (c) | T-829A; |
| (d) | G-792A; |
| (e) | T-775C; |
| (f) | C-532T; |
| (g) | G-217A; |
| (h) | C172T; |
| (i) | G384A; |
| (j) | G400A; |
| (k) | G507A; |
| (l) | A676G; |
| (m) | A698G; |
| (n) | A1035G; |
| (o) | A1164G; |
| (p) | C2079T; |
| (q) | G2624A; |
| (r) | A3189G; |
| (s) | T3965C(P199P); |
| (t) | A5093C; |
| (u) | C5343T; |
| (v) | G5556A; |
| (w) | G5593A; |
| (x) | A5878C; |
| (y) | A6066C; |
| (z) | G6152A; |
| (aa) | C6233T; |
| (ab) | G6309A; |
| (ac) | C6420T; |
| (ad) | C6428G; |
| (ae) | G6442A; |
| (af) | G7369A; |
| (ag) | C8357T; |
| (ah) | T9597C; |
| (ai) | G9669T; |
| (aj) | A9770G; |
| (ak) | C11535A; |
| (al) | C11608T; and |
| (am) | G12058A. |

11. A method for determining whether an individual has, or is predisposed to developing, hypertension associated with an AGT hypertensive haplotype, the method comprising analyzing at least part of the DNA sequence of the angiotensinogen (AGT) gene of said individual for the presence of an allelic pattern comprising at least two alleles wherein each allele comprises an SNP selected from the group consisting of:

| | |
|---|---|
| (a) | A-1178G; |
| (b) | G-1074T; |
| (c) | T-829A; |
| (d) | G-792A; |
| (e) | T-775C; |
| (f) | C-532T; |
| (g) | G-217A; |
| (h) | C172T; |
| (i) | G384A; |
| (j) | G400A; |
| (k) | G507A; |
| (l) | A676G; |
| (m) | A698G; |
| (n) | A1035G; |
| (o) | A1164G; |
| (p) | C2079T; |
| (q) | G2624A; |
| (r) | A3189G; |
| (s) | T3965C(P199P); |
| (t) | A5093C; |
| (u) | C5343T; |
| (v) | G5556A; |
| (w) | G5593A; |
| (x) | A5878C; |
| (y) | A6066C; |
| (z) | G6152A; |
| (aa) | C6233T; |
| (ab) | G6309A; |
| (ac) | C6420T; |
| (ad) | C6428G; |
| (ae) | G6442A; |
| (af) | G7369A; |
| (ag) | C8357T; |
| (ah) | T9597C; |
| (ai) | G9669T; |
| (aj) | A9770G; |
| (ak) | C11535A; |
| (al) | C11608T; and |
| (am) | G12058A, |

wherein the presence of said allelic pattern indicates that the individual is predisposed to the development of, or has hypertension.

12. The method of claim 11 wherein said predisposition is a predisposition to essential hypertension.

13. The method of claim 11 wherein said predisposition is a predisposition to pregnancy-induced hypertension.

14. The method of claim 11 wherein the genomic sequence of at least one allele of the AGT gene of said individual is analyzed.

15. The method of claim 11 wherein a part of the genomic sequence of at least two alleles of the AGT gene of said individual are analyzed.

16. The method of claim 11 wherein said analysis is performed by hybridization of at least one nucleic acid to the AGT gene of said individual.

17. The method of claim 16 wherein said hybridization is performed with an allele-specific oligonucleotide probe.

18. The method of claim 11 wherein said analysis is carried out by sequence analysis.

19. The method of claim 11 wherein said determination of the AGT gene is carried out by SSCP analysis.

20. The method of claim 11 wherein a part of the genomic sequence of at least one of said two alleles of the AGT gene of said individual is analyzed.

21. The method of claim 19 wherein said analysis is carried out by hybridization of a nucleic acid probe to at least one of said two alleles of the AGT gene.

22. The method of claim 19 wherein said analysis of at least one of said two alleles of the AGT gene is determined hybridization is with an allele-specific oligonucleotide probe.

23. The method of claim 11 wherein said analysis is carried out by SSCP analysis.

24. The method of claim 11 wherein a part of the genomic sequence of the AGT gene of said human is analyzed.

25. A method of determining the predisposition of an individual to hypertension which comprises analyzing at least part of the DNA sequence of the angiotensinogen (AGT) gene of said individual for the presence of at least one haplotype for the AGT gene, wherein said haplotype is selected from the group consisting of HA1, HA2, HA3, HA4, HA5 and HG1.

26. The method of claim 25 wherein said predisposition is a predisposition to essential hypertension.

27. The method of claim 25 wherein said predisposition is a predisposition to pregnancy-induced hypertension.

28. The method of claim 25 wherein the genomic sequence of at least one allele of said haplotypes for the AGT gene of said individual is analyzed.

29. The method of claim 25 wherein a part of the genomic sequence at least two alleles of said haplotypes for the A GT gene of said individual are analyzed.

30. The method of claim 25 wherein said analysis is performed by hybridization of at least one nucleic acid to the AGT gene of said individual.

31. The method of claim 30 wherein said hybridization is performed with at least one allele-specific oligonucleotide probe.

32. The method of claim 25 wherein said analysis is carried out by SSCP analysis.

33. The method of claim 25 wherein a part of the genomic sequence of at least one of two alleles of said haplotypes for the AGT gene of said individual is analyzed.

34. The method of claim 33 wherein said analysis is carried out by hybridization of a nucleic acid probe to at least one of two alleles of said haplotypes for the AGT gene.

35. The method of claim 25 wherein said analysis is carried out by sequence analysis.

36. The method of claim 24 wherein said analysis is carried out by SSCP analysis.
